(19) United States
(12) Patent Application Publication
(10) Pub. No.: US 2013/0280696 A1
(43) Pub. Date: Oct. 24, 2013

(54) DEVICES AND METHODS FOR DETECTING ANALYTE IN BODILY FLUID

(71) Applicants: Elliott MILLENSON, Far Hills, NJ (US); Wendy STRONGIN, Far Hills, NJ (US); Carolyn MILLENSON, Far Hills, NJ (US)

(72) Inventors: Elliott MILLENSON, Far Hills, NJ (US); Wendy STRONGIN, Far Hills, NJ (US); Carolyn MILLENSON, Far Hills, NJ (US)

(21) Appl. No.: 13/844,712

(22) Filed: Mar. 15, 2013

Related U.S. Application Data
(60) Provisional application No. 61/637,100, filed on Apr. 23, 2012.
(60) Provisional application No. 61/647,007, filed on May 15, 2012.
(60) Provisional application No. 61/667,739, filed on Jul. 3, 2012.

(51) Int. Cl. G01N 33/569; A61B 5/145

(52) U.S. Cl. CPC G01N 33/56988; A61B 5/14546
USPC 435/5; 435/287.2; 600/309

(57) ABSTRACT

A test device for detecting an analyte in a sample of bodily fluid, includes at least one hollow needle configured to obtain a sample of bodily fluid, an enclosure configured to contain a reagent, a portion configured to open the enclosure, a test results indicator configured to absorb the bodily fluid, the test results indicator including at least one test zone configured to indicate the presence of an analyte in the bodily fluid when the at least one test zone is in fluidic contact with the reagent, a housing, and a cover configured to be movable with respect to the housing between a closed position precluding access to the at least one hollow needle and an open position permitting access to the at least one hollow needle. Additionally, the at least one hollow needle is in fluid communication with the test results indicator.

### CROSS-REFERENCE TO RELATED APPLICATIONS

This application is based upon and claims the benefit of priority under 35 U.S.C. §119(e) of U.S. Provisional Application No. 61/637,100 to Millenson et al. filed on Apr. 23, 2012, U.S. Provisional Application No. 61/647,007 to Millenson et al. filed on May 15, 2012, and U.S. Provisional Application No. 61/667,739 to Millenson et al. filed on Jul. 3, 2012, the complete disclosures of which are incorporated by reference herein.

### FIELD OF THE INVENTION

Embodiments of the present invention relate generally to the fields of immunoassay and molecular diagnostic test devices and methods for detecting the presence of an analyte. More particularly, embodiments relate to test devices having reagents for performing the detection as a part of the device.

### BACKGROUND OF THE INVENTION

AIDS is responsible for the deaths of an estimated 30 million people since its discovery in 1981. Human immunodeficiency virus, or HIV, is the virus that causes AIDS. HIV/AIDS weakens a person's immune system and their ability to fight infections and cancer. While there is no HIV cure at this time, there are treatments that can delay the onset of AIDS, treat AIDS symptoms, and prevent pregnant women from passing HIV to an unborn fetus. A person may be infected with HIV without knowing it until they undergo HIV testing. HIV can be transmitted during protected and unprotected sexual relations, or via contact with certain bodily fluids of an infected individual.

Early detection of the virus is essential for improving the treatment outcomes and for the reduction of future transmission of the infection. Current HIV detection options include tests performed in clinical environments and “mail in” tests that are analyzed in a laboratory. There is a need for an HIV test-kit that can provide highly accurate and immediate results at the place and time of one's choosing, including in the home, in a hotel/motel room, in a bar, in a bath house, at a point-of-sex environment, or at a point-of-care environment.

### SUMMARY OF THE INVENTION

In accordance with some embodiments of the invention, a test device for detecting an analyte in a sample of bodily fluid may include at least one hollow needle configured to obtain a sample of bodily fluid. The test device may also include an enclosure configured to contain a reagent and a portion configured to open the enclosure. Further, the test device may include a test results indicator configured to absorb the bodily fluid, the test results indicator including at least one test zone configured to indicate the presence of an analyte in the bodily fluid when the at least one test zone is in fluidic contact with the reagent. In addition, the test device may include a housing, and a cover configured to be movable with respect to the housing between a closed position precluding access to the at least one hollow needle and an open position permitting access to the at least one hollow needle. Additionally, the at least one hollow needle may be in fluid communication with the test results indicator.

In various embodiments, the test device may include one or more of the following features: the test results indicator is configured to indicate the presence of HIV; the at least one hollow needle has a coating that comprises an anesthetic; the at least one hollow needle has a longitudinal length equal to or less than 4 mm; the test results indicator is a first test results indicator configured to indicate the presence of a first analyte in the bodily fluid, and the test device further includes a second test results indicator configured to absorb the bodily fluid, the second test results indicator includes at least one second test zone configured to indicate the presence of a second analyte in the bodily fluid when the at least one second test zone is in fluidic contact with the reagent, the second analyte differs from the first analyte, and the first test results indicator is spaced apart from the second test results indicator; a first fluid transport portion between the at least one hollow needle and the first test results indicator, and a second transport portion between the at least one hollow needle and the second test results indicator; the first fluid transport portion is configured to transport a larger volume of the bodily fluid than the second fluid transport portion; and the cover is configured to be in the open position while the enclosure is open.

In some embodiments, a test device for detecting an analyte in a sample of bodily fluid may include a sample collector defining a first volume configured to contain a sample of bodily fluid. The test device may also include an enclosure configured to contain a reagent and a portion configured to open the enclosure. Further, the test device may include a chamber defining a fixed second volume, the chamber being configured to permit mixing of the bodily fluid and the reagent. In addition, the test results indicator may be configured to absorb the bodily fluid, the test results indicator may also include at least one test zone configured to indicate the presence of an analyte in the bodily fluid when the at least one test zone is in fluidic contact with the reagent. Additionally, the sample collector may be in fluid communication with the test results indicator such that the bodily fluid flows from the sample collector to the test results indicator, and the sample collector may be configured such that the first volume remains substantially unchanged when bodily fluid flows from the sample collector to the test results indicator.

In various embodiments, the test device may include one or more of the following features: the test results indicator is configured to indicate the presence of HIV; the sample collector includes at least one hollow needle; the at least one hollow needle has a longitudinal length equal to or less than 4 mm; the sample collector includes an opening defining a cavity having an interior bottom portion, the interior bottom portion includes a raised section; the at least one hollow needle extends out from the bottom portion by a first height; the raised section has a second height greater than or equal to the first height; and at least a portion of the raised section is positioned between the at least one hollow needle and the opening.

Methods for detecting an analyte in a sample of bodily fluid are also disclosed. Some exemplary methods involve a test device including a sample collector defining a volume configured to contain a sample of bodily fluid, an enclosure containing a reagent, and a test results indicator configured to absorb the bodily fluid, the test results indicator including at least one test zone configured to indicate the presence of an analyte in the bodily fluid when the at least one test zone is in fluidic contact with the reagent. The method may also include acquiring the sample of bodily fluid with the sample collector, conveying the bodily fluid from the sample collector, releasing the reagent from the enclosure, and mixing the bodily fluid with the reagent. Further, the volume of the sample collector may remain substantially unchanged during the releasing the reagent.

In various examples, the method may include one or more of the following features: the sample collector includes at least one hollow needle, and acquiring the sample further includes piercing tissue via the at least one hollow needle such that the sample flows through the at least one hollow needle; the at least one hollow needle has a longitudinal length equal to or less than 4 mm; the test device further includes a cover and a housing, and the method further includes locking the cover to the housing after the acquiring the sample; the mixing the bodily fluid with the reagent occurs while the test device is positioned in any horizontal or vertical orientation; the acquiring the sample and the releasing the reagent occur at substantially the same time; and the method further includes indicating the presence or absence of the analyte in the bodily fluid.

Additional objects and advantages of the devices and methods will be set forth in part in the description which follows, and in part will be apparent from the description, or may be learned by practice of the devices and methods.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several exemplary embodiments of the invention and together with the description, serve to explain the principles of the invention.

FIG. 1 is a perspective view of an assay device according to an exemplary embodiment.

FIG. 2 is a perspective view of a portion of the assay device in FIG. 1.

FIG. 3 is a sectional side view of the assay device in FIG. 1.

FIG. 4 is a sectional side view of the portion of the assay device in FIG. 2.

FIGS. 5a and 5b are diagrams of an assay device according to another embodiment.

FIG. 6a is a diagram of an assay device according to another embodiment.

FIG. 6b is a diagram of an assay device according to another embodiment.

FIG. 7 is a diagram of an assay device according to another embodiment.

FIG. 8a is a perspective view of an assay device according to another embodiment.

FIG. 8b is a perspective view of the assay device in FIG. 8a with a microneedle matrix exposed.

FIG. 9 is a sectional side view of the assay device in FIG. 8a.

FIG. 10 is a block diagram of the assay device in FIG. 8a.

FIG. 11 is a sectional side view of an assay device according to another embodiment.

FIG. 12 is a perspective view of a portion of an assay device according to another embodiment.

FIG. 13 is a top-side perspective view of a cap and collector according to another exemplary embodiment.

FIG. 14 is a bottom-side perspective view of the collector in FIG. 13.

FIG. 15 is a sectional perspective view of another exemplary embodiment including the collector of FIG. 14 and a cap.

FIG. 16 is a sectional view of the cap and collector in FIG. 13.

FIG. 17 is a front view of the cap in FIG. 13.

FIG. 18 is a perspective view of a cap according to another exemplary embodiment.

FIG. 19 is a side view of a cap according to another exemplary embodiment.

FIG. 20 is a sectional view of a portion of a cap according to another exemplary embodiment.

FIG. 21a is a front sectional view of a portion of a cap according to another exemplary embodiment.

FIG. 21b is a side sectional view of the cap in FIG. 21a.

FIG. 22 is a sectional perspective view of a collector according to another exemplary embodiment.

FIG. 23a is a top view of a flow-through device according to another exemplary embodiment.

FIG. 23b is a front sectional view of the flow-through device in FIG. 11a.

FIG. 24a is a top view of a pouch-releaser according to another exemplary embodiment.

FIG. 24b is a side sectional view of the pouch-releaser in FIG. 24a.

FIG. 25a is a side sectional view of an ampoule-releaser according to another exemplary embodiment.

FIG. 25b is a front sectional view of the ampoule-releaser in FIG. 25a.

FIG. 25c is a perspective view of the ampoule-releaser in FIGS. 25a and 13b.

FIG. 26a is a side sectional view of an assay device according to another exemplary embodiment.

FIG. 26b is a side sectional view of an assay device in FIG. 26a.

FIG. 27a is a sectional perspective view of a sheath and microneedle matrix according to another exemplary embodiment.

FIG. 27b is a sectional perspective view of the sheath and microneedle matrix according to FIG. 27a.

FIG. 28 is a perspective view of an assay device according another embodiment.

FIG. 29 is a sectional perspective view of an assay device according another embodiment.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

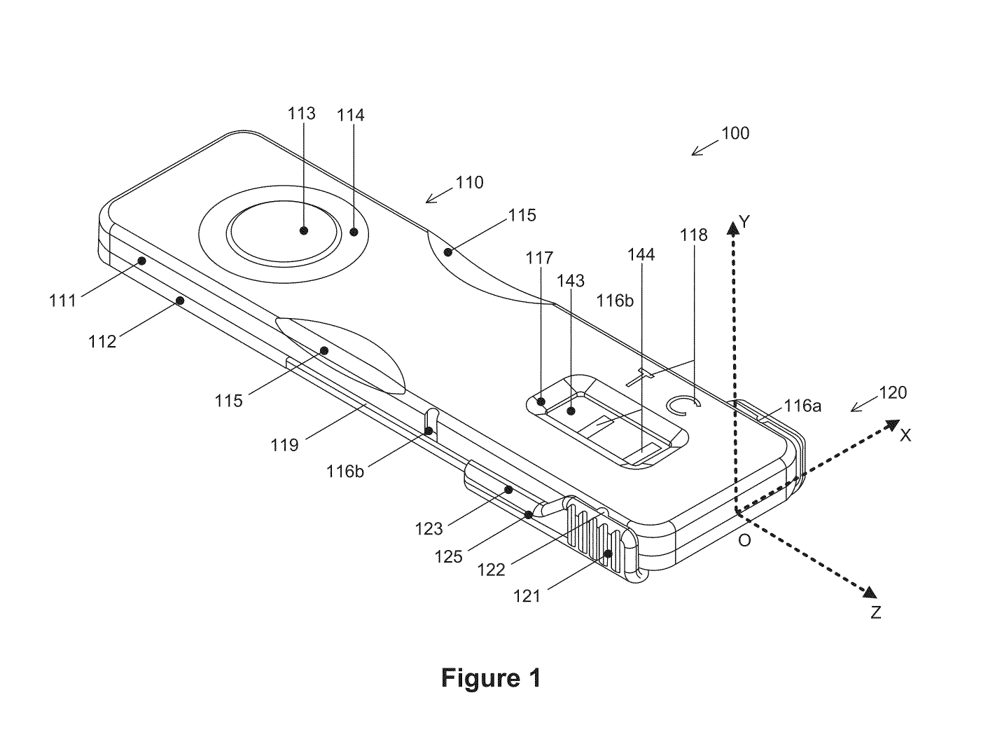

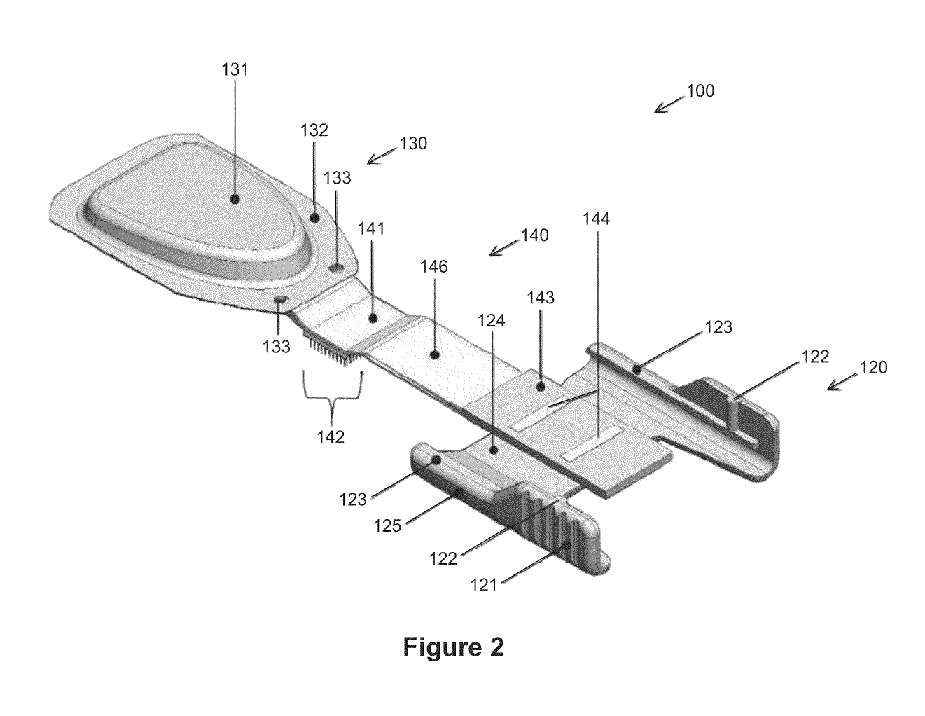

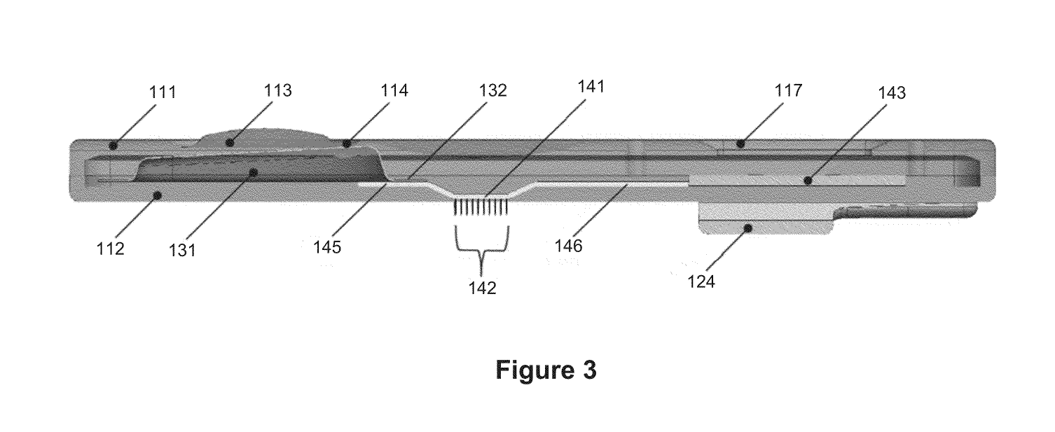

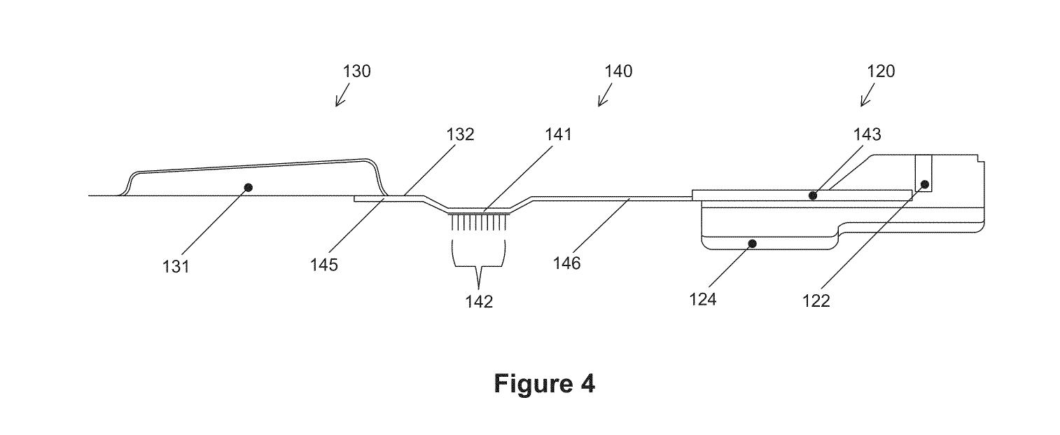

Turning now to the Figures, FIGS. 1-4 show an assay device 100 according to an exemplary embodiment. The assay device 100 may include a housing 110 having a substantially rectangular bar shape. In other embodiments, the assay device 100 may include a housing having a heart shape (not shown) as a symbol of sexual relations. The housing 110 may have a length in the direction of the Z-axis, a width in the direction of the X-axis, and a height in the direction of the Y-axis. The assay device 100 may have a proximal end including a datum point O at the vertex of the mutually perpendicular X-, Y-, and Z-axes as shown in FIG. 1. The assay device 100 may have a distal end located at a side opposite from the proximal end in the direction of the Z-axis.

The housing 110 may include thin walls having inner surfaces substantially defining a cavity in the form of a chamber with a substantially fixed volume. The housing 110 may include a top half 111 and a bottom half 112, with each half sharing a common perimeter defined by a plane orthogonal to the Y-axis. The top half 111 may include a top surface substantially orthogonal to the Y-axis.

The top half 111 may include an integrally formed button 113 near the distal end having a circular cross section when viewed in the direction of the Y-axis. The top of the button 113 may have a convex profile when viewed from the side or front in the direction of the X- or Z-axis respectively. The button 113 may be surrounded by an integrally formed deflector 114. The deflector 114 may include a weakened portion defined by a recess concentric with the button 113 and configured to deform either plastically or elastically and allow the button 113 to move in the direction of the Y-axis.

The top half 111 may include a window 117 located near a proximal end and defining a substantially rectangular opening when viewed in the direction of the Y-axis. The perimeter of the window 117 may be angled with respect to a plane orthogonal to the Y-axis such that the perimeter of the window 117 in successive planes from the outer surface towards the inner surface may decrease while maintaining the same aspect ratio. The window 117 also may include a transparent film or cover spanning across its opening.

The top half 111 may include a pair of grips 115, each of which may be defined by a recess symmetrical to a plane orthogonal to the X-axis. The grips 115 may be configured to be grasped by a hand or a portion of a hand to assist in positioning the assay device 100. The grips 115 also may facilitate providing relative movement of engageable elements such as the button 113 and/or a shield 120 with respect to the housing 110.

The top half 111 may include a plurality of stops 116a and stops 116b (collectively stops 116), each of the stops 116 may be defined by a recess having the shape of a truncated cylinder, with its axis parallel with the Y-axis. The stops 116 may be defined by a cross section of its perimeter orthogonal to the Y-axis having a semi-circular arc, in which each end point of the arc is coincident with a side of the top half 111 orthogonal to the X-axis. An equal number of the stops 116 may be located on opposite sides of the top half 111 symmetric to each other in the direction of the X-axis. In addition, stops 116a may be positioned near the proximal end of the housing 110, while stops 116b may be positioned distal to the stops 116a.

The top half 111 may include indicia 118 located near the window 117. The indicia 118 may be further positioned near test zones 144, and may provide identification representative of the test zones 144. The indicia 118 may include letters or symbols such as a “T” to designate a test portion of the test zones 144 and a “C” to designate a control portion of the test zones 144 as shown in FIG. 1. The indicia 118 may include engravings, embossed features, stickers, and/or paint.

The bottom half 112 may have substantially the same shape as the top-half 111 and may include a bottom surface opposite of the top surface of the top half 111. The bottom half 112 may include two sides parallel to a plane orthogonal to the X-axis, with each side defining a rectangular slot 119. Each of the slots 119 may include a top edge and a bottom edge or surface orthogonal to the Y-axis. The slots also may be symmetric to each other in the direction of the X-axis.

The shield 120 may slidably engage the housing 110 along a path from a proximal portion of the assay device 100 to a mid-portion thereof in the direction of the Z-axis. The shield 120 may include a side portion 125 having symmetrical vertical walls orthogonal to the X-axis and configured to remain outside of the housing 110. The side portion 125 may include grip portions 121 located towards the proximal end. Each of the grip portions 121 may include an array of repeating recesses, each of which may be aligned parallel with the Y-axis. The repeating recesses of the grip portion 121 may be evenly spaced and configured to be gripped by a finger or thumb. The shield 120 may include engaging portions 123, which may be integrally connected to the side portions 125 forming an inward projection orthogonal to the X-axis. The engaging portions 125 may be positioned within the slots 119 with a translational degree of freedom in the direction of the Z-axis. The shield 120 may include a cover portion 124, which may connect each of the side portions 125 outside of the housing 110 and beneath the bottom half 112. The shield 120 may include posts 122 integrally connected to or formed with an inward surface of the side portions 125 positioned external to the housing 110. The posts 122 may have substantially the same geometry of the slots 116 and may be configured to engage the slots 116 in a mating orientation.

The assay device 100 may include a reservoir or reservoirs 130 for holding a fluid or fluids. Hereinafter the terms “reservoir” and “fluid” will encompass their respective plural variations as well. The reservoir may be positioned within the housing 110 and beneath the button 113. The reservoir 130 may include a thin walled bladder 131 defining a cavity for holding a fluid comprising a reagent or reagents. Hereinafter the term “reagent” will encompass the plural form “reagents” as well. The reagent may comprise, for example, one or more of the following: enzymes, solvents, buffers, stabilizers, surfactants, salts, reducing agents, lysing agents, labels, proteins, antigens, antibodies and substrates. The reservoir 130 may include a flat base 132, a portion of which may form a bottom surface of the bladder 131, and a portion of which may form a lip extending in outward directions away from the bladder 131 in all directions orthogonal to the Y-axis. The outer perimeter of the base 132 may have substantially the same shape as the perimeter of the base 132 at its interface with the bladder 131. The bottom surface of the base 132 may be contiguous with the inner surface of the bottom half 112. The base 132 may include multiple circular edges proximal to the bladder 132 defining holes 133. The holes 133 may be symmetric to each other about the Z-axis and may be configured to attach or position the reservoir 130 to the bottom half 112 and/or a porous matrix 140. The base 132 may include a weakened surface beneath the bladder 131 located near the proximal end of the reservoir 130. The weakened surface may be configured to open or rupture if an internal pressure within the bladder 131 exceeds a predetermined amount.

The porous matrix 140 may be positioned between the reservoir 130 and the window 117. The porous matrix 140 may include a test results indicator 143 positioned beneath the window 117. The test results indicator 143 may include lateral flow and flow-through detection devices including test zones 144 visible beneath window 117 and orientated in the direction of the X-axis. The zones 144 may be separated in the direction of the Z-axis by non-indicating portions of the porous matrix 140. The test results indicator 143 may determine the presence and/or amount of an analyte in a liquid sample as the liquid sample moves through the assay device by lateral flow or capillary action. The assay device may be used in a vertical or a horizontal orientation or in an orientation between vertical and horizontal. Patents teaching lateral flow detection devices include U.S. Pat. Nos. 5,569,608, 5,120,643, 5,656,503, 4,855,240, and 5,591,645, British Patent GB 2204398A, and European Patent EP 0323605 B1, each of which is hereby incorporated by reference in its entirety.

The test results indicator 143 may contain materials configured to change color in the presence of an analyte. An analyte, for example, may be a compound or composition to be detected that is capable of binding to a ligand, receptor, enzyme, antibody, antigen, or nucleic acids. Analytes may include proteins, hormones, sugars, ligands, antibodies, antigens, RNA, DNA, viruses, viral particles and receptors, including active fragments or fragments thereof. An analyte may further include an analyte analogue, which is a derivative of an analyte, such as, for example, an analyte altered by chemical or biological methods, such as by the action of reactive chemicals, such as adulterants, enzymatic activity, or pH modifiers. A non-exhaustive list of analytes may include, for example, cholesterol, low density lipoprotein cholesterol (LDL cholesterol), high density lipoprotein cholesterol (HDL cholesterol), triglycerides, glucose, iron, ferritin, prostate specific antigen (PSA), creatine phosphokinase (CPK), CPK-BB, CPK-MB, CPK-MM, luteinizing hormone, human chorionic gonadotropin (HCG), α-HCG, β-HCG, and hepatitis specific antibodies. The test results indicator 143 may also contain materials configured to change color in the presence of other components in a reagent or sample.

The porous matrix 140 may include a combined transport portion 146 located distal to and in fluidic communication with the test results indicator 143. The porous matrix 140 may contain a microneedle matrix 142 distal to the combined transport portion 146. The microneedle matrix 142 may include one or more hollow needles, each of which is substantially aligned in the direction of the Y-axis and may traverse a portion of the bottom half 112. Each of the needles in the microneedle matrix 142 may have a tip forming an angle with the Y-axis. The tip angle may be between 0 to 75 degrees and may further be between 0 to 20 degrees. Each microneedle may include an opening at the apex of the tip, or the opening may be offset from the apex of the tip to prevent clogging from skin during penetration. For example, the apex of the tip may have a closed end and an opening may be located on a side wall in relatively close proximity to the apex. Additionally, each of the needles may have a longitudinal length equal to or less than 4 mm, e.g., in the range of from about 100 to about 4000 microns or a range of from about 500 to about 1500 microns. Further, each of the needles may have an effective base diameter between about 150 to about 400 microns. Any suitable size microneedle may be used, however, to obtain the desired amount of sample. The microneedle matrix 142 may have a needle density of about 10 to about 1000 needles per square centimeter, though more or less needles may be used depending on the amount of sample that is to be obtained.

An open end of each needle may be located outside of the housing 110, while an opposite end of each needle may be located within the cavity of the housing 110 terminating at a sample collection portion 141. In addition, the microneedles may have a coating that includes a topical anesthetic, such as lidocaine. However, other anesthetics may be used, such as procaine, amethocaine, benzocaine, tetracaine, prilocalne, bupivicaine, levobupivacaine, ropivacaine, mepivacaine, dibucaine and etidocine. The coating may also include one or more biodegradable polymers. For example, a biodegradable polymer may be a polysaccharide, such as dextran. The coating may also include an adjuvant, such as epinephrine bitartrate to prolong the local residence of the anesthetic in bodily tissue. In some embodiments, an anesthetic may be provided with the assay device in a separate container, dispenser, or package. For example, an anesthetic may be provided in a spray dispenser, vial, dropper, or wipe, and may be applied directly to the microneedle matrix 142 prior to use.

The sample collection portion 141 may be located distal to and in fluid communication with the combined transport portion 146. The sample collection portion 141 also may be in fluidic communication with the microneedle matrix 142 and a reservoir transport portion 145. The reservoir transport portion 145 may be located distal to the sample collection portion 141, and may include a portion located beneath or proximally to the weakened surface of the base 132.

In some examples, a user at a point-of-care or a point-of-sex location may wish to perform an assay test to determine if he/she or potential sex partner(s) will test positive/negative for HIV 1, HIV 2, or any associated subtypes. An assay test may utilize an antibody, antigen, or nucleic acid probe/fragment to specifically bind to an analyte. Patents teaching an assay test include U.S. Pat. Nos. 4,313,734, 4,446,232, 4,806,311, 4,868,108, 6,300,068, 6,875,572 and 7,541,194, each of which is hereby incorporated by reference in its entirety.

The user may remove the assay device 100 and read an instruction set and safety precautions (not shown) from a packaging container (not shown). The assay device 100 may be packaged with the shield 120 in the covered position. In the covered position, the posts 122 may be positioned within the stops 116b, such that the tips of the microneedle matrix 142 are unexposed. In the covered position, accidental contact with and damage to the microneedle matrix 142 is reduced. The user may grasp the housing 110 along the grips 115 with one of his/her hands and slide the shield 120 to the open position. In the open position, the posts 122 may be positioned within the stops 116a, such that the tips of the microneedle matrix 142 are exposed. To move the shield 120 from the closed position to the open position, the user may first apply pressure to the distal end of the grip portion 121 and bias the posts 122 out of the stops 116b and then slide the shield 120 in the direction of the Z-axis to the position where the posts 122 become seated within the stops 116a. Once the shield 120 is in the open position, the user may direct the assay device 100 to a position on the surface of his/her body or someone else's body. The user may urge the assay device 100 against the skin until the microneedle matrix 142 punctures the skin. As the microneedle matrix 142 punctures the skin, the microneedles substantially retain their shape and the capacity (interior volume) of the microneedles remains substantially unchanged. After puncturing the skin, the user may hold the microneedle matrix 142 against the skin for a period of time, or the user may immediately withdraw the microneedle matrix 142 from the skin while keeping the tips of the microneedles in fluidic contact with bodily fluid on the skin. The sample of bodily fluid may be wicked-up through the microneedle matrix 142 to the sample collection portion 141 by capillary pressure. A sample may include, for example, whole blood, serum, plasma, and interstitial fluid.

The user may depress the button 113 to compress the reservoir 130 between the button 113 and the inside of the bottom half 112. Once the pressure inside the bladder 131 exceeds a predetermined amount, reagents contained within the bladder are exuded onto the reservoir transport portion 145. The reagents may be released prior to, at the same time, or after the collection of the sample. The reagents may include enzymes such as horseradish peroxidase, alkaline phosphatase, f3-galactosidase, substrates such as TMB (3,3′,5,5′tetramethylbenzidine), DAB (3,3′,4,4′diaminobenzidine), 4CN (4-chloro-1-naphthol), TMB (dual function substrate), ABTS (2,2′-azino-di [3-ethylbenzthiazoline]sulfonate), OPD (o-phenylenediamine), BCIP/NBT (5-bromo-4-chloro-3-indolyl-phosphate/nitroblue tetrazolium) p-NPP (p-nitrophenylphosphate), a control antibody and additionally, solvents/surfactants to facilitate the transport of the enzymes, antibodies and substrates within the interstitial fluid to the test results indicator 143. Driven by capillary pressure, the reagents may flow from the reservoir transport portion 145 to the sample collection portion 141, and the sample may flow from the microneedle matrix 142 to the sample collection portion 141 as well. The reagents and the sample may flow from the sample collection portion 141 to the test results indicator 143 through the combined transport portion 146. The reagents and the sample may flow through the combined transport portion 146 along the same pathway to permit mixing, or the reagents and the sample may flow through substantially different portions of the combined transport portion 146 to substantially prevent mixing prior to reaching the test results indicator 143.

One of the test zones 144 may include an immobilized HIV-specific antigen such as P24, a control antigen, and other control compounds such as a paired HIV-specific antibody/antigen. If an HIV-specific antibody is present in the collected fluid, it may bind to the HIV-specific antigen and the associated enzyme may react with the conjugate pair to produce a color indication of the presence of the HIV-specific antibody. At a different test zone not shown, the enzyme may react with a paired HIV-specific antibody/antigen to produce a color indicating a positive control. At an even different test zone (not shown), a control antibody may bind to a control antigen and an associated enzyme may react with the conjugate pair to produce a color indicating a negative control. Based upon the indications from the positive and/or negative controls, the user may then acknowledge that the test results are ready. The positive control may indicate the condition in which the enzyme has traveled to a positive control zone with sufficient strength to react with an antibody/antigen conjugate pair, and further indicate the appearance of a positive result for comparison. Whereas, a negative control may indicate the appearance of a negative test result for comparison as well.

An antibody may refer to, for example, an immunoglobulin that specifically binds to and is thereby defined as complementary with a particular spatial and polar organization of another molecule. The antibody can be monoclonal or polyclonal and can be prepared by techniques that are well known in the art such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′).sub.2, Fab′, and the like. In addition, aggregates, polymers, and conjugates of immunoglobulins or their fragments can be used where appropriate so long as binding affinity for a particular molecule is maintained.

An antigen may refer to a compound or composition that generates or captures antibodies. An antigen may include recombinant protein, synthetic peptide, virus or a viral lysate. Antigens associated with HIV may include gp 160, env gp 120, env gp 41, gag p17, gag p24, gp 36, and ANT70.

The housing 110 and the shield 120 may be constructed with various materials and techniques and may be comprised of a metal or a plastic such as acrylonitrile butadiene styrene (ABS), polypropylene (PP), polycarbonate (PC), polystyrene (PS), acetal, styrene-acrylonitrile (SAN), acrylic, nylon, polyester, polyetherimide (PEI), polyphenylene sulfide (PPS), polyether ether ketone (PEEK), polyphenylene oxide (PPO), polyethersulfone (PES) polybutylene succinate, thermoplastic elastomers (TPE), acrylo-styrenic, ethylene, liquid crystal polymer (LCP), polyaryl, polybutylene, polycrylic, polyether, polyethylene (PE), polymethyl, polyphenylene, polypropylene, polyurethane, polyvinyl, styrenic, phenolic, melamine, epoxy, PTFE (polytetrafluoroethylene), and diallyl phthalate (DAP). The above mentioned elements may be extruded, machined, or molded, including injection molded. The elements may be integrally connected to each other, separate elements or groups of elements assembled together. The reservoir 130 and the microneedle matrix 142 may be comprised of a plastic such as those mentioned above, glass, silicon polymers or a metal, including metallic films, such as aluminum.

The porous matrix 140 and the test results indicator 143 may comprise absorbent material that has a configuration to absorb bodily fluid, including, but not limited to, PE, PP, polyvinylidene fluoride (PVDF), PTFE, polyester, nylon 6 (N6) and PES. The porous matrix and test indicator materials may either be themselves hydrophilic (so as to readily uptake bodily fluids) or be treated (i.e., with a surfactant/detergent) so as to be hydrophilic.

The reagent may include other labels such as magnetic beads (e.g., Dynabeads™), fluorescent dyes (e.g., fluorescein isothiocyanate, Texas Red, rhodamine, green fluorescent protein, and the like), radiolabels (e.g. ³H, ¹²⁵I, ³⁵S, ¹⁴C, or ³²P), colorimetric labels such as colloidal gold, gold conjugates, silver, selenium, or other metals, or colored glass or plastic (e.g., polystyrene, polypropylene, latex, etc.) beads, chemiluminescent labels, and avidin-biotin. Patents teaching the use of such labels include U.S. Pat. Nos. 3,817,837, 3,850,752, 3,939,350, 3,996,345, 4,277,437, 4,275,149, and 4,366,241, each of which is hereby incorporated by reference in its entirety. Means of detecting such labels are well known to those having skill in the art. Thus, for example, radiolabels may be detected using photographic film or scintillation counters. Fluorescent markers may be detected using a photodetector to detect emitted illumination. In some embodiments, the reservoir 130 may be positioned distal to the microneedle matrix 142 and the test results indicator 143, or the reservoir 130 may be positioned between the microneedle matrix 142 and the test results indicator 143. When two reservoirs are included in the assay device 100, both reservoirs may be positioned distal to the microneedle matrix 142 and the test results indicator 143, or both reservoirs may be positioned between the microneedle matrix 142 and the test results indicator 143. Also, one the reservoirs may be positioned distal to the microneedle matrix 142 and the test results indicator 143, while the other reservoir may be positioned between the microneedle matrix 142 and the test results indicator 143.

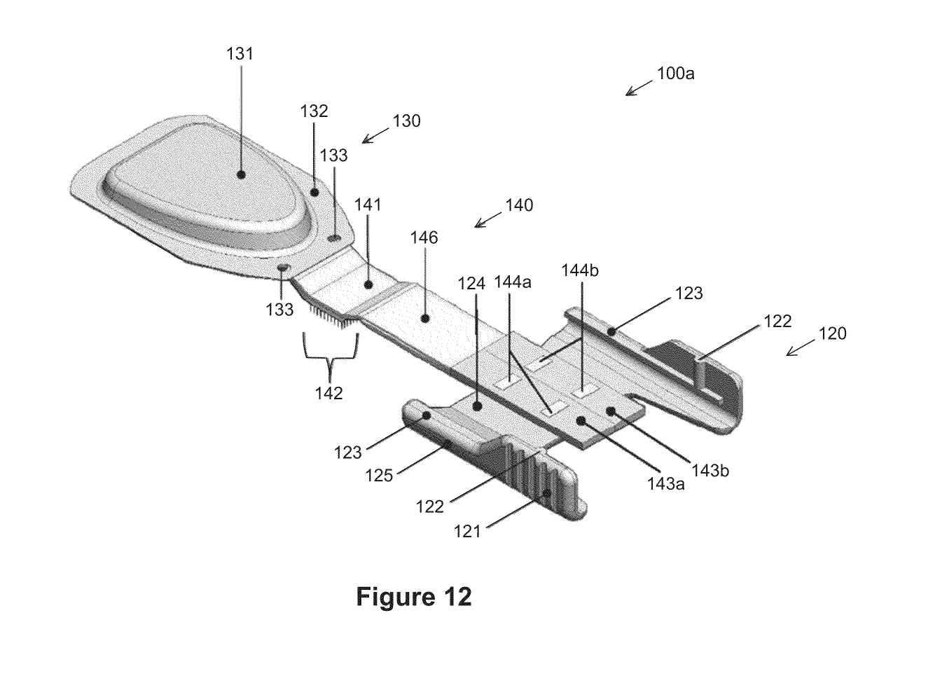

In another embodiment shown in FIG. 12, the test results indicator 143 may include a partition, such as a complete incision or partial incision (not shown), bifurcating or separating the test results indicator 143 into multiple test results indicators 143a and 143b. The test results indicators 143a and 143b may include test zones 144a and 144b configured to test different analytes. In addition, the top half 111 may include multiple windows (not shown) with each window corresponding to each of the test zones 144a and 144b. Having two different test results indicators may allow a user to test for different analytes, for example, HIV antigen and HIV antibody. Also each of the test results indicators 143a and 143b may include separate test and control analytes at each of the test zones 144a and 144b. Optimal chemical testing conditions for HIV antigen and HIV antibody may not necessarily be the same for both tests. For example, it may be advantageous for each to have a different pH. In some embodiments, the volume of blood entering the multiple test strips may be different. The cross-sectional area of test results indicators 143a and 143b in fluidic contact with combined transport portion 146 may be different by a predetermined amount. In other embodiments, the combined transport portion 146 may also include an incision (not shown) dividing it into multiple sections corresponding to each of the test results indicators 143a and 143b. Each section of the incised transport portion may have different widths or thicknesses, and may be configured to transport different volumes of fluid compared to each other. Each section of the incised transport portion may be in fluid communication with different portions of the microneedle matrix 142. For example, half of the microneedles in the microneedle matrix 142 may be in fluid communication with one section of the incised transport portion while the other half of the microneedles in the microneedle matrix 142 may be in fluidic communication with the other section of the incised transport portion.

In other embodiments, the microneedle matrix 142 may be connected to a plurality of tubes (not shown) terminating at the combined transport portion 146. Each of the tubes may correspond to a single microneedle or to multiple microneedles and may have different diameters with respect to each other. In some embodiments, a single tube (not shown) may connect the microneedle matrix 142 to the combined transport portion 146. In some embodiments, the tube may be bifurcated with each end terminating at an incised combined transport portion (not shown). Also, the diameters of the bifurcated portions of the tubes may be different with respect to each other.

In another embodiment, the volume of the collected sample may be controlled. The assay device 100 may include a collection chamber positioned between the sample collection portion 141 and the microneedle matrix 142. The collection chamber may further include a window for optical inspection of the sample collected. The assay device 100 may have a valve positioned between either the collection chamber and the microneedle matrix 142 or the collection chamber and the sample collection portion 141. The valve may reduce or prevent uptake of the sample, while in the closed position. The valve may be configured to open with the actuation of button 113. The valve may include a plunger extending within the collection chamber. The valve also may be configured to open by drawing the plunger out of the collection chamber, thus creating an expansion in volume and a partial vacuum to assist in withdrawing a sample.

In other embodiments, the top half 111 may include a transparent material, and may obviate the need for a window to view the test results indicator 143. In this embodiment, the test results may be visible through the transparent top half 111.

In other embodiments, a reservoir including a reagent may be integrally molded with the housing 110.

In other embodiments, the bottom half 112 may include a pointed or sharp element protruding from its inner surface. The pointed element may be located beneath the reservoir 130 to assist in opening the bladder of the reservoir 130, when the bladder is urged into it.

In other embodiments, the test results indicator 143 may include circuit elements capable of measuring a quantifiable degree of a positive result. This quantifiable degree may be compared to predetermined levels to label the result into categories. The test results indicator may further include a display such as an LED array or LCD to reveal the label of the result. The label may be a characterizing word or phrase representative of the category and may include “positive,” “negative,” “retest,” “borderline,” “uncertain,” “indeterminate,” and/or “see your doctor.” The display may include backlighting to assist the user in reading the results in a poorly lit environment.

In other embodiments the reservoir 130 may be opened with an electric signal such as an actuator. The reservoir 130 may also be opened by a change in temperature such as electrically heating a wire embedded within a heat sensitive membrane, such as gelatin membrane.

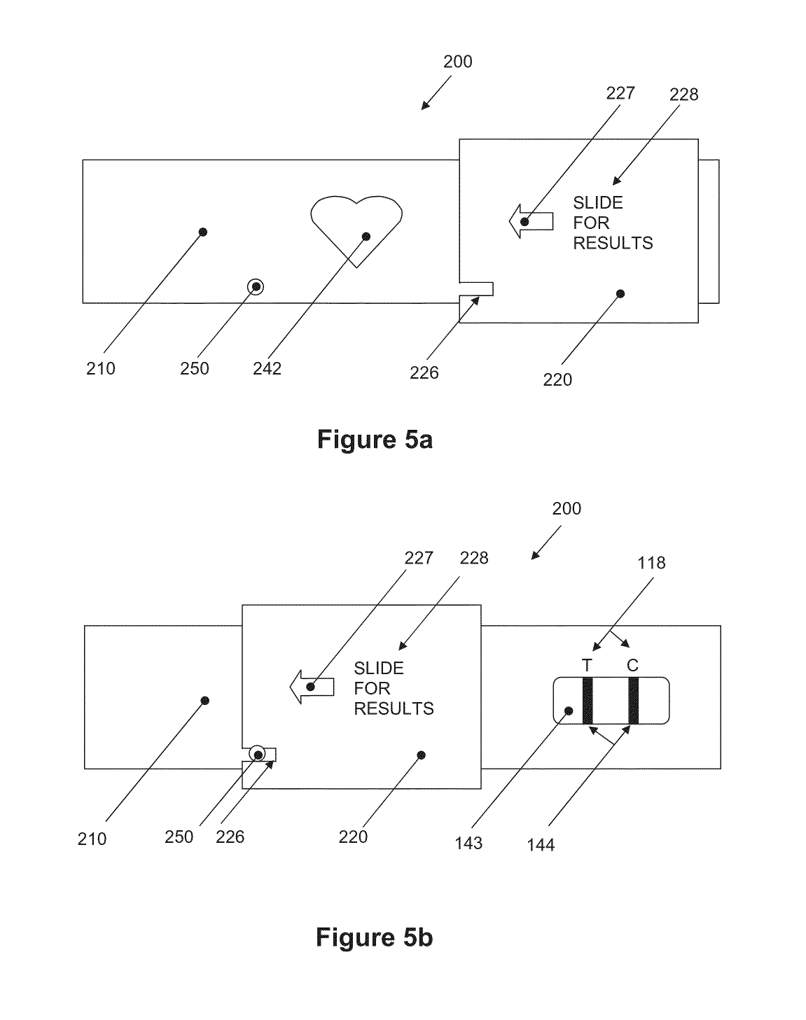

In another embodiment shown in FIGS. 5a and 5b, an assay device 200 may include substantially the same elements and coordinate system as the assay device 100 shown in FIGS. 1-4 and described above. However, the assay device 200 may include a shield 220 instead of shield 120. Shield 220 may include a continuous surface defining two open ends. The shield 220 may be configured to slideably engage a housing 210 instead of housing 110 in the direction of the Z-axis. The shield may include a first locking mechanism 226 configured to lock with a second locking mechanism 250 included within or on the housing 110. The locking mechanisms 226 and 250 are respectively diagrammed as a pin and a slot in FIGS. 5a and 5b, however in other embodiments the “pin” or male locking portion may be located on the shield 220 and the “slot” or female locking portion may be located on the housing 210. Each of the locking mechanisms 226 and 250 may include surfaces configured to substantially allow engagement in one direction, and elastic portions configured to hold the shield 220 in the locked position, as shown in FIG. 5b. For example, the pin may be formed of an elastic material and have a diameter slightly larger than the width of the slot. When the slot receives the pin, the elastic material will permit the diameter of the pin to decrease in the direction of the slot's width, and the slot will securely receive the pin by a friction fit. The locking mechanisms 226 and 250 may be located external to the housing 210 or inside the housing 210, to prevent accidental unlocking. FIG. 5a demonstrates the shield 220 in the unlocked position. In this configuration, a microneedle matrix 242 may be uncovered and have unimpeded access to it. The footprint of microneedle matrix 242 may be shaped like a heart, or other curved shapes and may include soft color tones to be more inviting to users. The shield 220 may include indicia 227 and 228 to provide instructions to the user. The indicia 227 may include an arrow for example, while indicia 228 may include the words “Slide for Results.” The indicia 227 and 228 may include engravings, embossed features, stickers, and/or paint.

After a sample has been acquired with assay device 200 in substantially the same method as the assay device 100 described above, additional steps may be performed, including locking the shield 220 into a position covering the microneedle matrix 242. The user may follow the instructions provided by the indicia 227 and 228 to reveal the test results on the test results indicator 143. In the process of sliding the shield 220 in the direction shown by indicia 227, the first locking mechanism 226 may engage the second locking mechanism 250, thereby locking the shield 220 into a position covering the microneedle matrix 242. The lock formed by the engagement of the locking mechanisms 226 and 250 with each other may be permanent and may prevent accidental needle stick from the microneedle matrix 242. The assay device 200 may include a release mechanism (not shown) to release the first locking mechanism 226 from the second locking mechanism 250. The release mechanism may include a recessed button accessible by a small diameter pin, such as the tip of a paper clip. Alternatively, the release mechanism may be an exposed button. The release mechanism may be utilized if a user prematurely locks the first and second locking mechanisms 226 and 250 before a sample has been acquired.

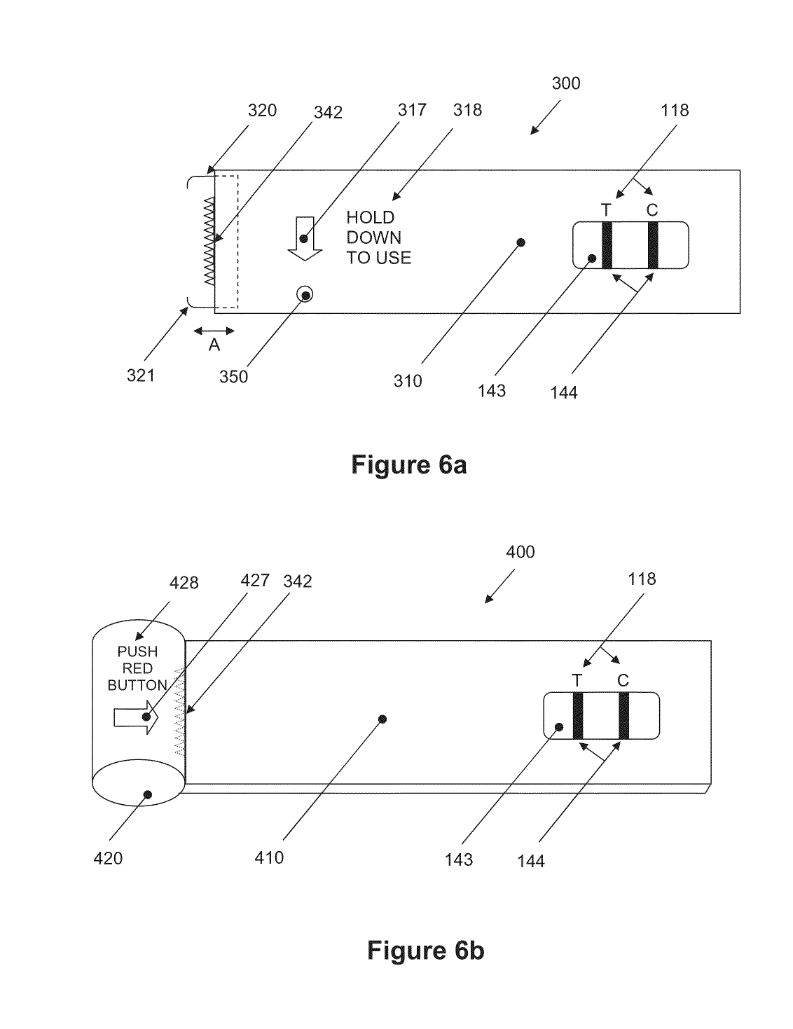

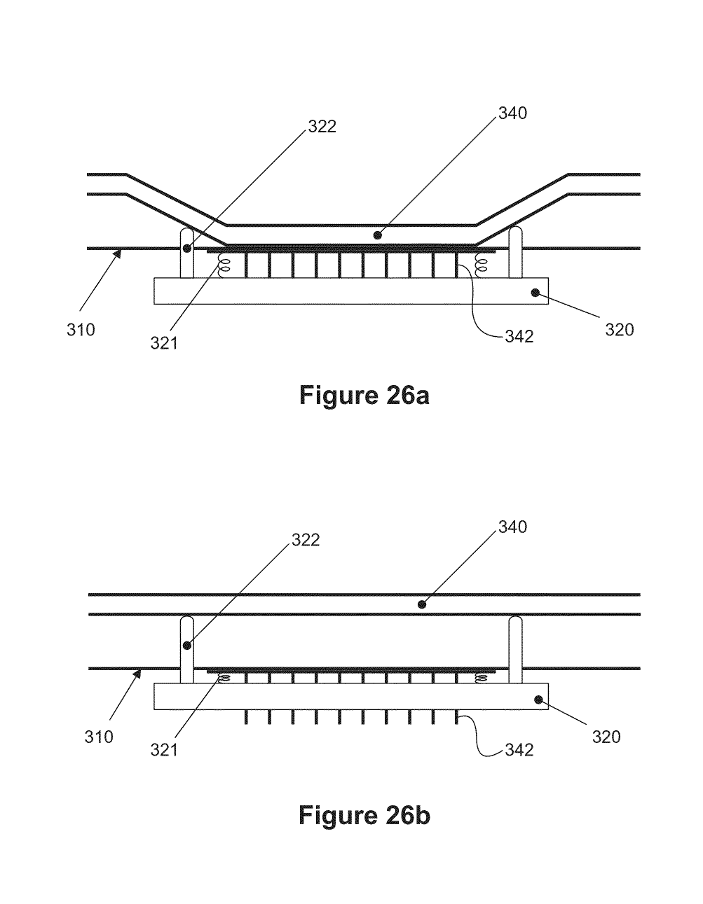

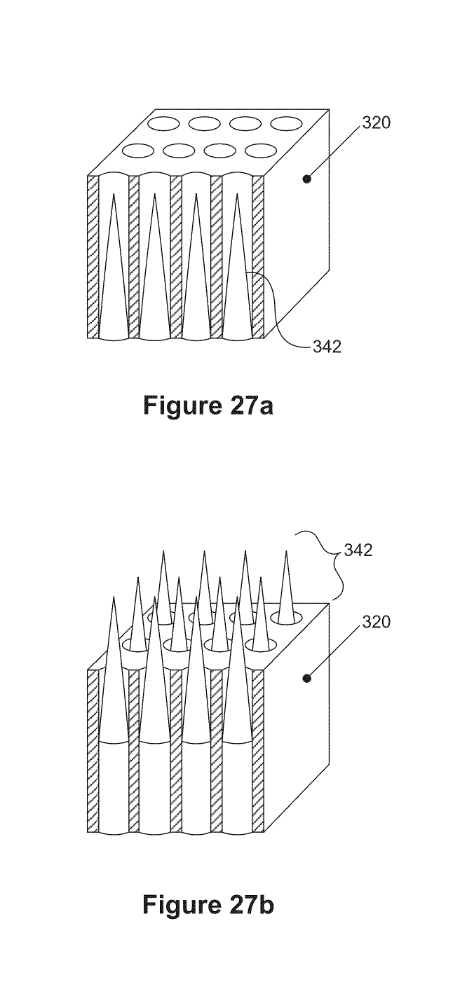

In another embodiment shown in FIGS. 6a and 26a-27b, an assay device 300 may include substantially the same elements and coordinate system as the assay device 100 shown in FIGS. 1-4 and described above. However, the assay device 300 may include a sheath 320 instead of shield 120. In some embodiments, the assay device may include both sheath 320 and a shield similar to shield 120. The sheath 320 may include an open-ended enclosure substantially forming four walls aligned with four walls of a housing 310. The sheath 320 may have portions extending external to and inside the housing 310 or the sheath 320 may be located substantially external to the housing 310. The sheath 320 may be connected to the housing 310 at the distal end as shown in FIG. 6a or at any other surface of the housing 310. The sheath 320 may be located in a position configured to surround a microneedle matrix 342 in orientation parallel with the base of the microneedle matrix 342. For example, the microneedle matrix 342 may be located on a bottom surface of the assay device 300 similar to assay device 100 (see FIGS. 26a and 26b). The microneedle matrix 342 may be substantially similar to microneedle matrices 142 or 242. The sheath 320 may be transparent, and may include individual channels surrounding each individual microneedle or smaller groups of microneedles. The transparency of the sheath 320 may reduce user anxiety by allowing the user to see and contrast the relative size of a microneedle to a traditional hypodermic needle. In the retracted position, a curved rim 321 of the sheath 320 may abut the exterior of housing 310 to act as a stop for the sheath 320. The sheath 320 may be configured to move in the direction of the Z-axis (as shown by arrow A) from a fully extended position outside of the housing 310 to a retracted position within the housing 310. The sheath 320 may further include one or more springs 321, internal to housing 310 for example, to bias the sheath 320 in the extended position under a zero-load condition covering the microneedle matrix (see FIGS. 26a and 27a). For example, the sheath 320 may extend outward from the housing 310, and retract towards the housing 310 when an external load in the Z-direction is applied to it, exposing the microneedle matrix 342 (see FIGS. 26b and 27b). The housing 310 may include a locking button 350. The button 350 may be mechanically coupled to the sheath 320 through a mechanical linkage (not shown) and configured to lock the sheath 320 in the extended position. The button 350 may include a spring (not shown) to bias the button 350 in the extended position under a zero-load condition. The housing 310 may include indicia 317 and 318 to provide instructions to the user. The indicia 318 may include an arrow for example, while indicia 318 may include the words “Push to Use.” The indicia 317 and 318 may include engravings, embossed features, stickers, and/or paint. Optionally, the sheath 320 may include one or more posts 322 attached to a surface of the sheath facing the housing 310 and extending inward into the housing 310. When the sheath 320 is under a zero-load condition in the extended position, posts 322 may make contact with a porous matrix 340. The porous matrix 340 may be the same as or substantially similar to porous matrix 140. When the sheath 320 is forced inward, posts 322 exert a force on the porous matrix 340, and separate the porous matrix 340 from the microneedle matrix 342. When the sheath 320 returns to the extended position, the porous membrane 340 may reestablish contact with the microneedle matrix 342.

A user may engage the microneedle matrix 342 with tissue by positioning the distal surface of the sheath 320 against the targeted tissue. The user may follow the instructions provided by the indicia 317 and 318 to hold down the button 350. While holding the button 350 in the depressed position, the user may exert a force upon the assay device 300 in a direction substantially parallel to the longitudinal axes of the microneedles. The sheath 320 may then retract relative to the housing 310 exposing the microneedle matrix 342 to the tissue. Additional force may allow the microneedle matrix 342 to break the surface of the tissue and allow the acquisition of the sample. If, for example, a user attempts to retract the sheath 320 without holding down the button 350, the sheath 320 will remain in the extended position covering the microneedle matrix 342. Additionally, the sheath 320 may be mechanically coupled to a reservoir (not shown) to release reagents. The sheath 320 also may be mechanically coupled to a piston (not shown) for providing a vacuum to the microneedle matrix 342.

In another embodiment, the sheath 320 may be stationary with respect to the housing 310. The microneedle matrix 342 may be recessed with respect to the sheath 320. A button (not shown) may be mechanically coupled to the microneedle matrix 342. Actuation of the button may cause the microneedle matrix 342 to extend beyond the sheath 320, providing access to the microneedle matrix 342. Returning the button to its previous position may allow the microneedle matrix 342 to return to the recessed position.

In another embodiment shown in FIG. 6b, an assay device 400 may include substantially the same elements and coordinate system as the assay device 100 shown in FIGS. 1-4 and described above. However, the assay device 400 may include a sheath 420 instead of shield 120. The sheath 420 may be connected to a housing 410 instead of housing 110 at the distal end as shown or at any other surface of the housing 410. The sheath 420 may have the form of a thin-walled cylindrical vessel with one or two open ends. The microneedle matrix 342 may be substantially similar to microneedle matrices 142 or 242 and positioned through and within the sheath 420 along an interior surface. The sheath 420 may be transparent to reduce user anxiety by allowing the user to see and contrast the relative size of a microneedle to a tradition needle. The microneedle matrix 342 may include a color different from the color of the housing 410, such as red. The sheath 420 may include indicia 427 and 428 to provide instructions to the user. The indicia 427 may include an arrow for example, while indicia 428 may include the words “Push Red Button.” The indicia 427 and 428 may include engravings, embossed features, stickers, and/or paint.

A user may follow the instructions provided by indicia 427 and 428 to engage the microneedle matrix 342. A user may insert a finger into the sheath 420 and while the user's finger is inside the sheath 420, press down on the microneedle matrix 342 from inside the sheath 420, with the expectation of pressing a red button. Pressing down on the microneedle matrix 342 may allow a sample to be acquired from the user's finger. Further, the sheath 420 may be mechanically coupled to a reservoir (not shown) to release reagents. The sheath 420 also may be mechanically coupled to a valve (not shown) to provide a vacuum to the microneedle matrix 342.

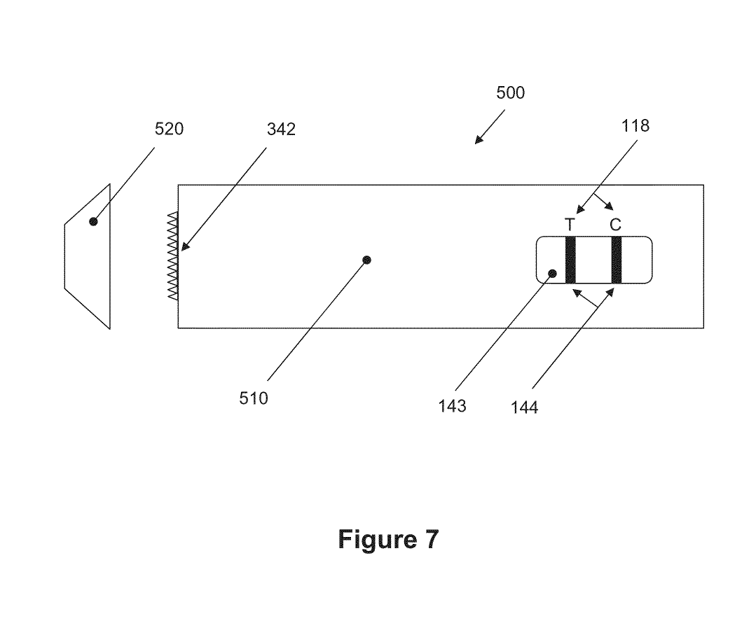

In another embodiment shown in FIG. 7, an assay device 500 may include substantially the same elements and coordinate system as the assay device 100 shown in FIGS. 1-4 and described above. However, the assay device 500 may include a cap 520 which may include a reservoir (not shown). The assay device 500 may include a housing 510 similar to housing 110, however housing 510 may include a distal end configured to be inserted into the cap 520. The housing 510 may include the microneedle matrix 342 at the distal end of the housing. The housing 510 and the cap 520 may include locking elements (not shown) configured to permanently lock the housing 510 into the cap 520 and seal off access to the microneedle matrix 342.

A user may acquire a sample as discussed above by inserting the microneedle matrix 342 into the targeted tissue. After a sample has been acquired, a user may then insert the distal end of the housing 510 into the cap 520. The microneedle matrix 342 may then break the surface of the reservoir to collect a reagent. The insertion of the microneedle matrix 342 into the reservoir may pressurize the fluid within the reservoir to assist in both the collection of the reagent and the transport of the reagent and the sample to the test results indicator 143. At substantially the same time as the microneedle matrix 342 breaks the surface of the reservoir, the housing 510 may become permanently locked with the cap 520. Additionally, the housing 510 may include a sheath (not shown) similar to sheath 420, along with the locking button 350. Indicia may be included, providing instructions regarding the use of the locking button 350.

In other embodiments, a microneedle matrix may be located on a cap similar to cap 520. The microneedle matrix may be in fluid communication with a reservoir housed within the cap. A user may acquire a sample with the microneedles on the cap and then connect the cap to a housing similar to 510. At substantially the same time as the cap is inserted onto the housing, an urging member on a distal portion of the housing may pressurize the cap and a reagent from the reservoir may pass through the microneedles, into the housing, and to a test results indicator similar to test results indicator 143.

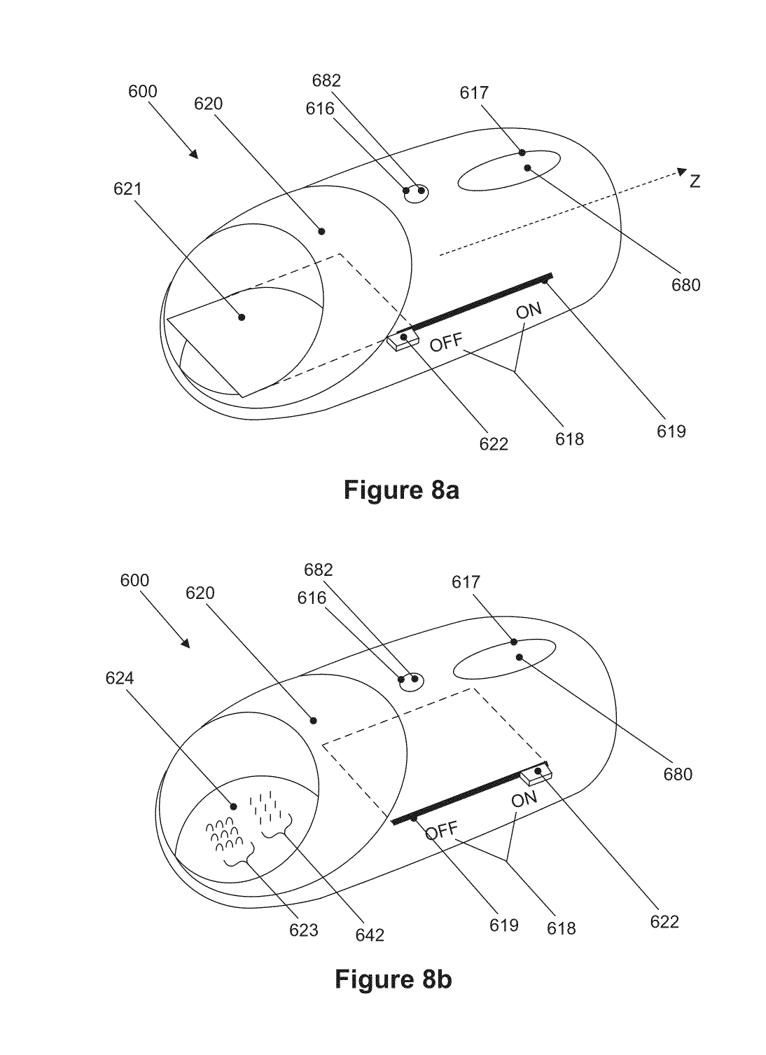

In another embodiment shown in FIGS. 8a-10, an assay device 600 may include a housing 610 having a bullet shape defining a cavity. The housing 610 may include a flat bottom surface 611 parallel to a longitudinal Z-axis, the bottom surface may provide stability for resting the assay device 600 on a flat surface. An end of the housing 610 may include a rounded or partially-spherical enclosed end, whereas an opposite end of the housing 610 may include baffle 613 having a generally sloping wall. A shield 620 may include a generally cylindrical shell, and share a contiguous edge with a perimeter of the baffle 613, and may define a circular or elliptical opening at an opposite end. The shield 620 may be transparent, whereas in other embodiments, the shield 620 may be opaque. In contrast, the baffle 613 may be opaque to prevent ambient light from entering the interior of the housing 610. The housing 610 may include an edge defining a display window 617. The display window 617 may allow viewing of display 680, which may be positioned beneath the display window 617. The housing 610 also may include an edge defining an opening 616. An LED 682 may be positioned within the opening 616. In other embodiments, multiple LEDs may be positioned in the opening 616, or multiple LEDs may be positioned in additional openings similar to opening 616.

Furthermore, the housing 610 may include a slit 619 extending in a direction parallel with the Z-axis. A tab 622 may extend from the inside of the housing 610 outwardly through slit 619. The tab 622 may be integrally connected to a cover 621. The cover 621 may be coplanar with the tab 622 and may have a flat rectangular shape. In other embodiments, the cover 621 may be curved or elliptical when viewed from above. The tab 622 may be connected to the cover 621 and be configured to be gripped by a finger or fingernail. The tab 622 may slideably engage the slit 619 in a direction parallel with the Z-axis. The housing 610 may include indicia 618 including the words “On” and “Off.” The word “Off” may be located beneath the slot 619 towards the end of the slot 619 nearest the shield 620. Additionally, the word “On” may be located beneath the slot 619 towards the end of the slot 619 opposite the word “Off.” The cover 621 may be configured to cover a microneedle platform 624 when the tab 622 is positioned above the word “Off” (see FIG. 8a) and the cover 621 may be configured to be situated within the housing 610 when the tab 622 is positioned above the word “On” (see FIG. 8b).

The microneedle platform 624 may include a generally flat surface parallel with the bottom surface 611. The microneedle platform 624 may be located near the open end of the shield 620 and may intersect the shield 620 and the baffle 613 to define a perimeter around the microneedle platform 624. The shield 620 and the microneedle platform 624 may also define a cavity having an opening providing access to a microneedle matrix 642. The microneedle matrix 642 may be located on the microneedle platform 624 near the interface of the microneedle platform 624 and the baffle 613. The microneedle matrix 642 may be the same as or substantially similar to microneedle matrix 142. One or more raised sections (e.g., bumps) 623 may extend out of the microneedle platform 624 between the microneedle matrix 642 and the opening of the shield 620. The raised sections 623 may extend above the microneedle platform 624 by a height greater than or equal to the height that the microneedles extend above the microneedle platform 624. The raised sections 623 may be positioned to substantially shield the microneedle matrix 642 from lateral access, thus the raised sections 623 may guide a user's finger to a position above the microneedle matrix 642; at which point, the user may contact the microneedle matrix 642 with a downward motion.

A reagent reservoir 631, similar to bladder 131, may be located beneath the microneedle platform 624 and above a reagent actuator 675. The reagent actuator 675 may include a linear actuator having a tip configured to puncture reagent reservoir 631 upon receiving an electric signal. The reagent actuator 675 may be electrically connected to a processor 686 and/or a power supply 685. The reagent actuator 675 may be located within a sample collection chamber 641. The sample collection chamber 641 may include a vessel defining a cavity, which has a volume defined by its outer walls. The sample collection chamber 641 may be in fluid communication with the reagent reservoir 631, the microneedle matrix 642, a test results indicator 643, and a vacuum 673. In particular, the sample collection chamber 641 may have an opening to allow fluid to flow to a test results indicator 143. The test results indicator 143 may include absorbent material disposed in the opening, such that fluid flowing through the opening comes in contact with, and flows at least partially through, the test results indicator 143. The vacuum 673 may be configured to provide a negative pressure differential inside the sample collection chamber 641 with respect to the outside of the assay device 600 upon receiving an electric signal. The vacuum 673 may be electrically connected to the processor 686 and/or the power supply 685. The test results indicator 643 may be the same as or substantially similar to test results indicator 143. The test results indicator 643 may extend outside of the sample collection chamber 641 to a position within the cavity of the housing 610. The test results indicator may further include one or more test zones (not shown), which are the same as or similar to test zones 144, positioned in close proximity to a light source 677 and a signal detector 676. The light source 677 may include multiple light sources and may be configured to emit single- or multi-wavelength light. Accordingly, the signal detector 676 may be configured to detect single- or multi-wavelength light as well. The signal detector 676 may be configured to detect the direct transmission and/or reflection of the light emitted from the light source 677. The light source 677 and the signal detector 676 may be electrically connected to the processor 686 and/or the power supply 685.

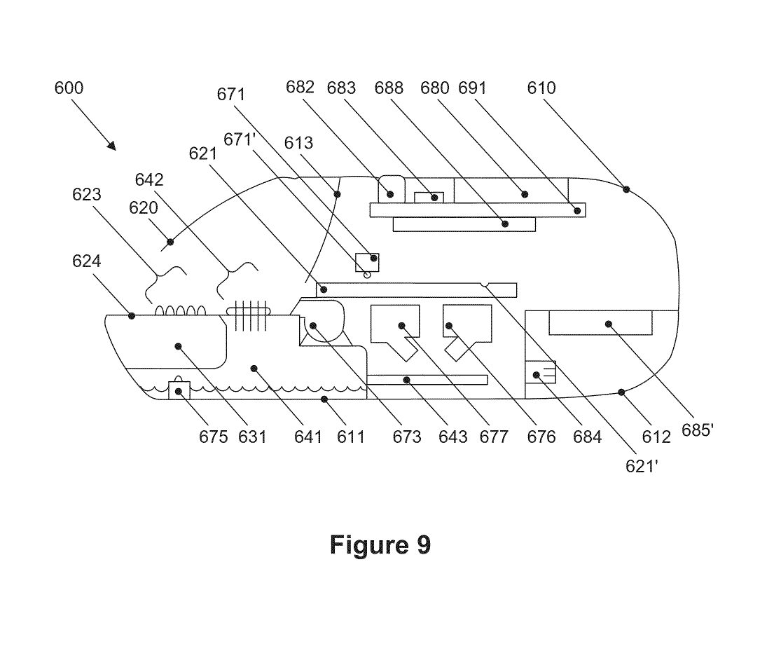

As shown in FIG. 9, the housing 610 may include a removable covering 612, which may further include a portion of the bottom surface 611 and a portion of the enclosed end of housing 610. The removable covering 612 may be configured to be repeatably removed and attached from the housing 610. The removable covering 612 may provide access to the inside of housing 610, and also may provide access to a battery 685′ and an input/output communication port (I/O Comm) 684. The battery 685′ may be configured to be removable and may include a non-conductive film (not shown) positioned between a terminal of the battery and an electrical contact from the power supply 685. Further, a portion of the non-conductive film (not shown) may extend from inside the housing 610 to outside of the housing 610 and may be configured to be removed while the removable covering 612 is attached to the housing 610. The I/O Comm 684, may be include a female or male connector configured to connect to an external communication device (not shown). The I/O Comm 684 may be electrically connected to the processor 686 and configured to transmit and receive data to and from the processor 686.

The cover 621 may substantially horizontally divide the interior of the housing 610 when in the on position. The cover 621 may be positioned above the vacuum 673, light source 677, and the signal detector 676. A locking actuator 671 may be positioned above the cover 621. The locking actuator 671 may include a linear actuator having a tip 671′ configured to interface with a notch 621′ located within the cover 621 upon receiving an electric signal. The locking actuator 671 may be electrically connected to the processor 686 and/or the power supply 685.

A printed circuit board (“PCB”) 691 also may be located above the door 621 and near the top of the interior of the housing 610. The LED 682 and the display 680 may be mounted on a surface of the PCB 691 and positioned to coincide with the opening 616 and the display window 617 as discussed above. The LED 682 and the display 680 may be electrically connected to the processor 686 and/or the power supply 685. In addition, a buzzer 683 may be mounted on the PCB 691 and may also be electrically connected to the processor 686 and/or the power supply 685. The processor 686 and the power supply 685 may be mounted on the opposite side of the PCB 691. The processor 686 may include a circuit and or various components of a circuit including a CPU 688, a timer 689, and a memory 690. The processor 688 may also include other circuit elements (not shown) for signal conditioning, converting analog signals to digital signals, converting digital signals to analog signals, amplifying signals, and various other processes. The processor 686 may also include a system bus 687. The system bus 687 may include a platform for data transfer to and from the processor 686. The system bus 687 may include a controller bus (collectively shown as the system bus 687) to directly provide/modulate power to some of the electrical components of the assay device 600. In addition, the controller bus 687 may control the power distribution from the power supply 685 to some of the electrical components of the assay device 600. The system bus 687 also may include an address bus (collectively shown as the system bus 687) to transfer data to and from the memory 690. The power supply 685 may include a circuit and or various components of a circuit including a battery 685′, voltage regulators (not shown), transformers (not shown), and AC signal generators (not shown).

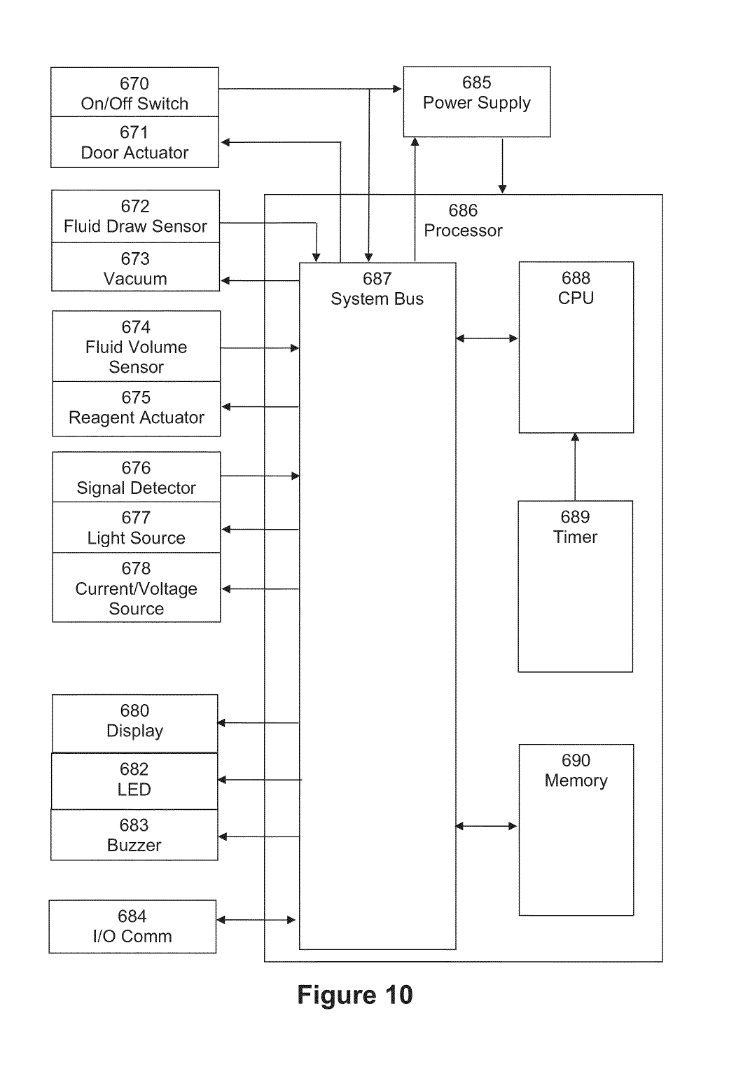

Referring to FIG. 10, the operation of the assay device 600 will be described. The assay device 600 may be powered by turning on an on/off switch 670. The on/off switch 670 may include electrical leads (not shown) configured to come into contact with each other when the tab is moved into the on position. When the electrical leads are in contact with each other, a circuit may be closed allowing the power supply 685 to power on at least the processor 686. The LED 682, display 680, and/or buzzer 683 may signal that the assay device 600 is ready to be used for a test. The user may then press down on the microneedle matrix 642 with his/her finger. As the user applies pressure to the microneedle matrix 642, blood may flow from his/her finger through the microneedle matrix 642 to the sample collection chamber 641. The flow of blood into and through the microneedle matrix 642 may be caused by capillary pressure and/or blood pressure, and may be further assisted by gravity if the orientation of the microneedles in the microneedle matrix 642 form an angle of less than 90 degrees with respect to the vector force of gravity. A fluid draw sensor 672 may indicate to the processor 686 that blood is being collected. The fluid draw sensor 672 may include a pressure sensor (not shown) coupled to the microneedle matrix 642, or may include electrical leads (not shown) positioned within the path of the blood flow and configured to become a closed circuit when in mutual contact with a conductive fluid, such as the blood. When the processor 686 is notified that blood draw has been initiated, the processor 686 may then turn on the vacuum 673. The vacuum 673 may move air from the sample collection chamber 641 to the outside of the assay device 600, thus creating a vacuum within the sample collection chamber 641 with respect to atmospheric pressure. As blood collects within the sample collection chamber 641, a fluid volume sensor 674 may measure the volume of blood collected. The fluid volume sensor 674 may include a pressure sensor (not shown) coupled to the base of the sample collection chamber 641, or may include electrical leads (not shown) positioned at a predetermined height above the base of the sample collection chamber 641 and configured to become a closed circuit when in mutual contact with a conductive fluid, such as the blood. When the processor 686 is notified that the blood volume has reached a predetermined volume, the processor 686 may then turn off the vacuum 673. Furthermore, when the blood has reached the predetermined volume, the processor 686 may turn on the reagent actuator 675 to puncture the reagent reservoir 631 and release reagents into the sample collection chamber 641. In other embodiments, when the blood has reached the predetermined volume, the processor 686 may initiate a predetermined delay, measured by the timer 689, to turn on the reagent actuator 675 to puncture the reagent reservoir 631 and release reagents into the sample collection chamber 641. The processor 686 may provide a signal to the LED 682, display 680, and/or buzzer 683 to indicate that a sufficient amount of blood has been collected.

In the sample collection chamber 641, the reagents and the blood may combine and mix together through diffusion. After a sufficient amount of blood has been collected, the reagents and the blood may be wicked onto a test results indicator 643. The processor 686 may turn on the light source 677 and signal detector 676, after the predetermined volume of blood has been collected. The signal detector 676 may output a voltage representative of an intensity of a detected wavelength or a range of wavelengths. Additionally, the signal detector 676 may be configured to detect the presence of multiple analytes. This may be done by having multiple signal detectors, or by multiplexing multiple signals into a single output at discrete moments in time. Furthermore, the signal detector 676 may be configured to detect multiple signals in the same sample at the test zones, multiple signals at multiple test zones on the test results indicator 643, or multiple signals at test zones on multiple test results indicators 643 as described above in reference to test results indicator 143. The processor 686 may then monitor the output from the signal detector 676 and apply a predetermined algorithm stored in the memory 690 to determine the presence of an analyte, positive control, and/or a negative control. The processor 686 also may use an input from the timer 689 to determine a length of time in which the test results indicator is monitored. After a positive, negative, or indeterminate result has been determined by the processor 686, the processor may signal to the LED 682, display 680, and/or buzzer 683 that a result has been displayed. In addition, the display 680 may display the results of the test. The results of test may be stored in the memory 690. After the processor displays the test results to the display 680, the processor 686 may turn on the door actuator 671 to bias the tip 671′ outward, and provide instructions to the text display 681 for the user to move the tab 622 into the off position. As the user moves the tab 622 into the off position, the tip 671′ may interface with the notch 621′ resulting in the door 621 locking in the off position. In other embodiments, cover 621 may be biased by a spring (not shown) into the off position when either the tab 622 is not held in the on position, or when a user's finger is positioned over the microneedle matrix 642. After the cover 621 has been locked in the off position, the processor 686 may cause the assay device 600 to operate in a low power consumption mode. In the low power consumption mode, a minimum amount of power may be drawn to display the test results indefinitely until power in the battery 685′ is depleted, or the battery 685′ is removed.

In another operation of the device, an algorithm may be uploaded into memory 690 by connecting a communication device (not shown) to the I/O Comm 684. The processor 686 may be configured to detect when a communication device is connected to the I/O Comm 684 and change the operating mode to a send and receive mode in which data may be either extracted or uploaded. Additionally, the assay device 600 may be powered by the communication device, when connected to the I/O Comm 684.

In another embodiment, a separate on/off switch or button (not shown) may be used to turn the assay device 600 on and off. In this embodiment, the operation of the on/off switch or button is not necessarily coupled to the operation of the cover 621.

In another embodiment, a signal detector may include a mechanical input such as a mechanical vibration. The mechanical vibration may be provided by a piezoelectric crystal or ceramic (not shown). Further, the mechanical vibration may be generated by exciting the piezoelectric crystal or ceramic with an alternating current generated by a current voltage source 678. The signal detector may detect frequency shifts associated with a reaction with an analyte and output a signal to the processor 686 representing the frequency shift.

In another embodiment, a signal detector may include a microfluidic device (not shown) configured to output a signal representative of the presence of an analyte.

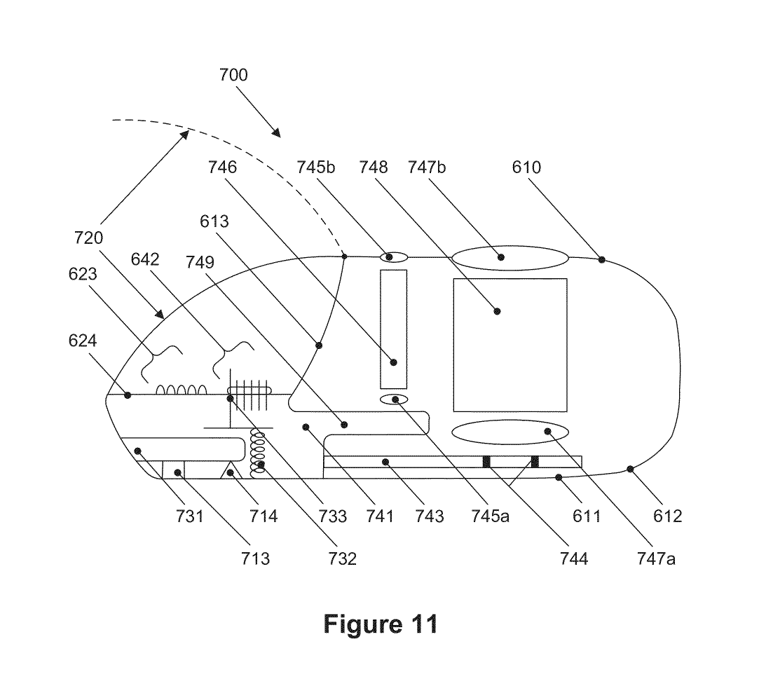

In another embodiment shown in FIG. 11, an assay device 700 may include substantially similar components noted with the same reference designators previously described in assay device 600. However, elements shown in FIG. 11 that are not common with assay devices 600 will be described herein, along with the operation of assay device 700. The assay device 700 may include a reagent reservoir 731 that may be opened manually, for example, the microneedle matrix 642 may include a plunger 733 biased away from the reagent reservoir 731 by a spring 732. The plunger 733 may include a shaft orthogonally connected to a flat surface. The shaft portion may extend upward beyond the microneedle platform 624. Beneath the reagent reservoir 731, a puncturing element 714 may be located. The puncturing element 714 may include a sharp tip located in close proximity to the reagent reservoir 731. Further, the puncturing element 714 may be located substantially below the plunger element 733, with the tip of puncturing element 714 in vertical alignment with the longitudinal axis of the shaft of the plunger 733. A support element 713 also may be located between the reagent reservoir 731 and the bottom of the housing 611. Alternatively, the puncturing element 714 may by located on the bottom surface of the plunger 733, and may be configured to puncture the reagent reservoir 731.

The assay device 700 may include a shield 720 in the form of a cover pivotally connected to housing 610. The shield 720 may be configured to rotate from a closed position (shown in FIG. 11 as a solid line) to an open position (shown in FIG. 11 as a dashed line), and also from the open position to the closed position. In the closed position, a user's access to the microneedle matrix 642 may be precluded, whereas in the open position, a user may access the microneedle matrix 642 in a similar manner as with assay device 600. Alternative cover arrangements, such as a cover that is removable with respect to a housing, or slideable with respect to a housing, rather than being pivotally connected, are also possible.

The assay device 700 may include a sample collection chamber 741 that is the same as or substantially similar to sample collection chamber 641. The sample collection chamber 741 may include a sample adequacy chamber area 749 positioned above a test results indicator 743 and in fluid communication with the sample collection chamber 741. The test results indicator 743 may be the same as or substantially similar to test results indicator 143, and may include test zones 744 that are the same as or substantially similar to test zones 144. The sample adequacy chamber area 749 may be defined by a transparent enclosure positioned below a first lens 745a. The housing 610 may include a second lens 745b having an edge conformal to the opening 616. A first light guide 746 may be positioned between lenses 745a and 745b, and may be configured to direct light between lenses 745a and 745b. The test zones 744 may be positioned below a third lens 747a. The housing 610 may include a fourth lens 747b having an edge conformal to the display window 617. A second light guide 748 may be positioned between lenses 747a and 747b, and may be configured to direct light between lenses 747a and 747b.

Operation of assay device 700 now will be described. A user may slide shield 720 into an open position to access the microneedle matrix 642. The user may simultaneously press down on the microneedle matrix 642 and the plunger 733 with his/her finger. Pressing down on the microneedle matrix 642 may cause blood to flow out of the user's finger, into the hollow interior(s) of the needle(s) of the matrix 642, and into the sample collection chamber 741. Pressing down on the plunger 642 may cause the plunger to compress the spring 732 and to force the reagent reservoir 731 against the puncturing element 714. After a sufficient force has been exerted by the user, the puncturing element 714 opens the reservoir 731 by puncturing the reservoir 731 and reagents flow out of the reagent reservoir 731 and into the sample collection chamber 741. When blood and reagents fill the sample collection chamber 741, the mixture of blood and reagents will start to fill the sample adequacy chamber area 749. At this point, a user may view the mixture of blood and reagents by looking into the opening 616. Optical detection of the mixture of blood and reagents may be amplified by lenses 745a and 745b. The optical pathway from the opening 616 to the sample adequacy chamber area 749 may include colored filters (not shown) to filter a bandwidth of red wavelengths associated with the color of oxygenated blood. The test results indicator 743 and the test zones 744 may operate in the same or substantially similar manner to test results indicator 143 and the test zones 144 as previously described. A user may view the test results indicated by the test zones 744 by looking through the display window 617. Optical detection of the test results may be amplified by lenses 747a and 747b. Removable covering 612 may be removed to insert or remove the test results indicator 743. In other embodiments, the mixing of the reagents with blood may be delayed until a sufficient quantity of blood has been collected.

In another embodiment, the sample collection chamber 741 and/or the sample adequacy chamber area 749 may include channels (not shown) to guide the flow of blood and/or reagents within the respective chambers. In addition, the sample collection chamber 741 and/or the sample adequacy chamber area 749 may include a porous matrix (not shown) to guide the flow of blood and or reagents within the respective chambers. Alternatively, the sample adequacy chamber 749 may be omitted and a dedicated portion of the test results indicator 643 may be used to indicate a sufficient sample. In this configuration, the first lens 745a may be positioned in proximity to the test results indicator 643.

In another embodiment, the reagent reservoir 731 may be punctured by an operation not directly coupled to pressing down on the microneedle matrix 642. For example, pressing a separate button (not shown) may allow a user to puncture the reagent reservoir 731 in a similar operation as described in the previous embodiments.

In other embodiments, elements included within assay device 600 may be combined with assay device 700. For example, an embodiment may include manual release of reagents from a reagent reservoir similar to reagent reservoir 731 in assay device 700 and may also include the electronic circuitry and associated elements described in connection with assay device 600 to display the test results on a digital display.

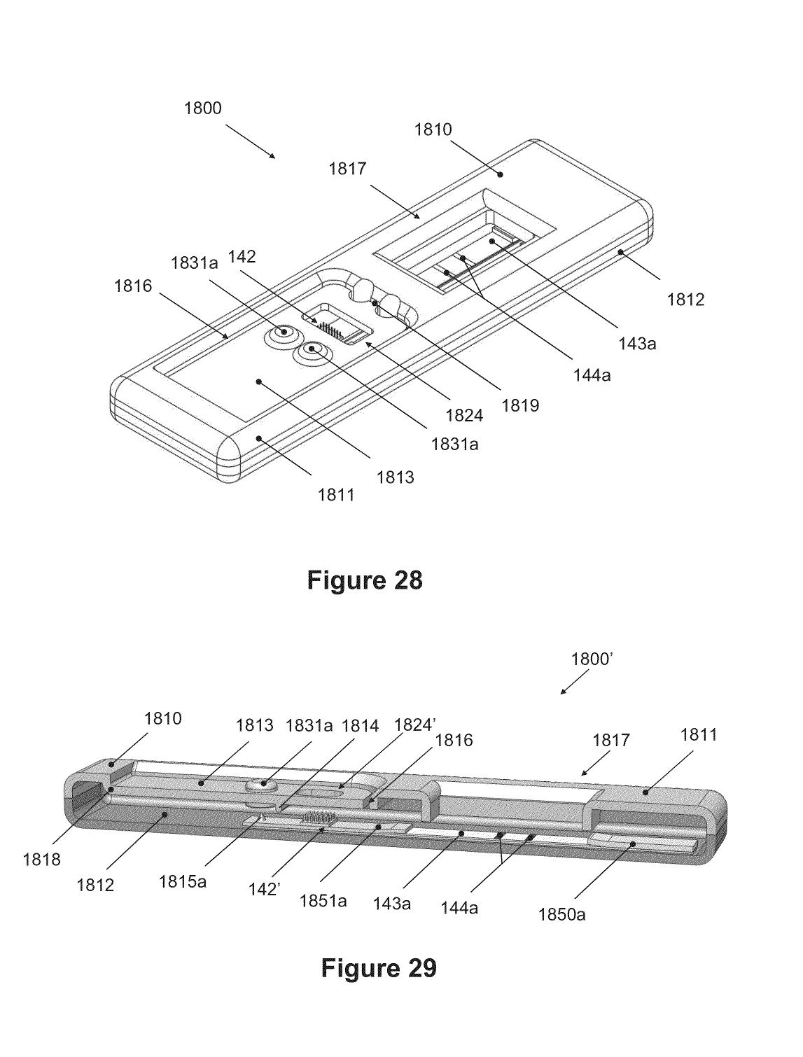

In another embodiment shown in FIG. 28, an assay device 1800 may include the same or substantially the same elements and coordinate system as the assay device 100 shown in FIGS. 1-4 and described above. However, the assay device 1800 may include a housing 1810 instead of housing 110. The housing 1810 may include thin walls having inner surfaces substantially defining a cavity in the form of a chamber with a substantially fixed volume. The housing 1810 may include a top half 1811 and a bottom half 1812, with each half sharing a common perimeter defined by a plane orthogonal to the Y-axis. The top half 1811 may include a top surface substantially orthogonal to the Y-axis.

The top half 1811 may include two edges defining two symmetric channels 1816 with respect to the Z-axis. Each of the channels 1816 may have a distal portion parallel to one another and the Z-axis. Each of the channels 1816 may also have a collinear-medial portion that is parallel to the X-axis. The medial portion of each of the channels 1816 terminates at a connecting portion 1817. The connecting portion 1817 may be a part of the top half 1811.

The top half 1811 may also include a lever portion 1813. The lever portion 1813 may be recessed with respect to the top half 1811, sharing at least a portion of the edges of the channel 1816. The lever portion 1813 may include a base portion 1818 which connects the lever portion 1813 to the top half 1811. The lever portion 1813 may also be connected to the top half 1811 by the connecting portion 1819, the base portion 1818 being distal to, and opposite of the connecting portion 1819. The lever portion 1813 may include a continuous edge defining an access port 1824. When viewed from above in the Y-direction, the continuous edge may have a shape such as a circle, an ellipse, an oblong circle, or a rectangle. The access port 1824 may be a through-hole in the lever portion 1813, providing access to the cavity formed by the housing 1810.

The lever portion 1813 may also include reservoirs 1831a and 1831b (collectively referred to as 1831) at a distal position with respect to the access portion 1824, and configured to contain a reagent. The reservoirs 1831 may be comprised of the same or similar materials as reservoir 130. The reservoirs 1831 may be integral with the lever portion 1813, or they may be separate elements. The reservoirs 1831 may extend in the Y-direction above the lever portion 1831 by a height, such that the top surfaces of the reservoirs 1831 are substantially at the same height as the top surface of the top half 1811. The reservoirs 1831 may also extend to a height, such that the top surfaces of the reservoirs 1831 are recessed with respect to the top half 1811. In addition, the reservoirs 1831 may extend in the Y-direction below the lever portion 1831 into the cavity formed by the housing 1810. The bottom surface of the reservoirs 1831 may include an enclosure bottom (not shown) that is configured to be punctured. For example the enclosure bottom may be comprised of a foil or film, or any same or similar material as bladder 131.

The lever portion 1813 may also include a restrictor portion 1814 positioned between the reservoirs 1831 and the access port 1824. The restrictor portion 1814 may be integral with the lever portion 1813 and may extend in the Y-direction from a bottom surface thereof into the cavity defined by the housing 1810. The restrictor portion 1814 may have the same width in the X-direction as the lever portion 1813. The restrictor portion 1814 may include angled sides with respect to the Y-direction, such that at least two of the angled sides intersect at its lowest point, away from the lever portion 1813 in the Y-direction.

The top half 1811 may include a window 1817 located near a proximal end and defining a substantially rectangular opening when viewed from above in the Y-direction. The window 1817 may include a transparent film or cover spanning across its opening, or may be left uncovered.

On the surface of the bottom half 1812, test results indicators 143a and 143b (collectively referred to as 143) may be held in place by distal attaching elements 1851a and 1851b (collectively referred to as 1851) and proximal attaching elements 1850a and 1850b (collectively referred to as 1851). The proximal attaching elements 1850 may be attached to the bottom half 1812 along a proximal portion of the proximal attaching elements 1850. The distal portion of the proximal attaching elements 1850 may hold a distal portion of the test results indicators 143 against the bottom half 1812. The distal attaching elements 1851 may hold in place, and cover a distal portion of the test results indicators 143. The microneedle matrix 142 may be positioned directly below the access port 1824 and on a portion of each of the distal attaching elements 1851a and 1851. Puncturing elements 1815a and 1815b (collectively referred to as 1815) may extend out of the distal attaching elements 1851. The puncturing elements 1815 may be positioned directly below the reservoirs 1813. Further, each of the puncturing elements 1815 may have a cone or pyramid shape and may be configured such that the sides of each of the puncturing elements 1815 form a point at its tip.

The test results indicators 143a and 143b may include respective test zones 144a and 144b (collectively referred to as 144). The test zones 144 may be positioned under the window 1817, so as to be visible from above. The microneedle matrix 142 may be in fluid communication with the test results indicators 143. In particular, some of the microneedles may be in fluid communication with the test results indicator 143a, whereas the remainder of the microneedles may be in fluid communication with the test results indicator 143b. In addition, the distal attaching elements 1851 may be comprised of permeable materials or may include passageways (not shown) to allow fluid to flow through it. The test results indicator 143a, distal attaching elements 1851a, and proximal attaching elements 1850a may be spaced apart from and symmetric to test results indicator 143b, distal attaching elements 1851b, and proximal attaching elements 1850b along an axis parallel to the Z-axis.

During operation of the assay device 1800, a user may place his/her finger over the access port 1824 and exert a force on the lever portion 1813. Once a sufficient force exerted on the lever portion 1813 exceeds a predetermined amount of force, the lever portion 1813 may break apart from the connecting portion 1819. The user may continue to urge the lever portion 1813 downward until his/her finger makes contact with and is pierced by the microneedle matrix 142. The base portion 1818 may flex as the lever portion 1813 is urged downward. At substantially the same time that the microneedle matrix 142 pierces the user's finger, the puncturing elements 1815 may puncture the reservoirs 1831, causing reagents to be released. Also at substantially the same time that the microneedle matrix 142 pierces the user's finger, the restrictor portion 1814 may contact the distal attaching portions 1851, and exert a force on the test results indicators 143 beneath the attaching portions 1851 such that the flow of reagents beyond the restrictor portion 1814 is significantly impeded. As the user removes his/her finger from the microneedle matrix 142 and the access port 1824, the lever portion 1813 retreats towards its previous position. As the lever portion retreats upwards, the restrictor portion 1814 retreats as well, removing the pressure that it applied to the test results indicators 143, and the reagents may flow towards the test zones 144. Test results indicators 143 and test zones 144 operate in a manner as discussed above with respect to the embodiments in FIGS. 1-4.

In another embodiment shown in FIG. 29, an assay device 1800′ may include the same or substantially the same elements and coordinate system as the assay device 1800 shown in FIG. 28 and described above. The assay device may include a microneedle matrix 142′ instead of microneedle matrix 142. The microneedle matrix 142′ may be positioned under access port 1824′ instead of access port 1824. Access device 1800′, operates in substantially the same manner as access device 1800.

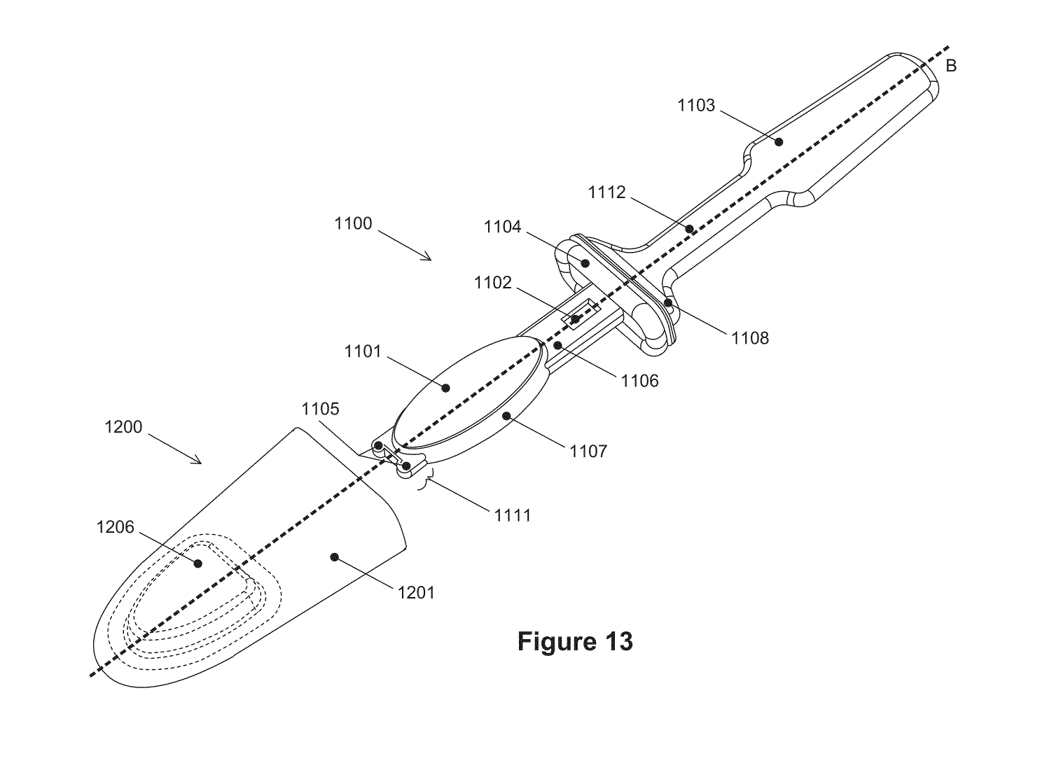

FIGS. 13-16 show a collector 1100 and cap 1200 according to another exemplary embodiment. In some embodiments the cap 1200 and collector 1100 are packaged separately in a box (not shown). In addition, the collector 1100 may be packaged inside a protective pouch or wrapper (not shown). The cap 1200 and collector 1100 may optionally contain an instruction set (not shown) within the box providing instructions for use and safety precautions.

The collector 1100 may be used to collect a sample and may include a handle 1103 and an absorbent pad 1101 to facilitate collecting the sample. In addition to whole blood, serum, plasma, and interstitial fluid, a sample may be comprised of saliva, oral fluid, urine, ocular fluid, semen, and/or spinal fluid. The handle 1103 and the absorbent pad 1101 may be located on substantially opposite ends of the collector 1100 along a generally longitudinal axis B and separated by a baffle member 1108. The end of the collector 1100 including the absorbent pad 1101 may be considered the distal end, whereas the end including the handle 1103 may be considered the proximal end. The handle 1103 may be configured to be gripped by a hand to move the collector 1100, and guide the absorbent pad 1101 towards a given target area. The target area may include for example, a subject containing a sample or the target area may include the cap 1200. Viewed from above, the handle 1103 may include a generally flat trapezoidal extrusion connected at its distal end to a rectangular neck 1112. The handle 1103 and the neck 1112 may each have a substantially rectangular cross section along an orthogonal plane to the longitudinal axis B, with the cross section of the neck 1112 including a smaller area at a longitudinal location. The neck 1112 may widen towards the middle of the collector 1100 to form a baffle 1108.

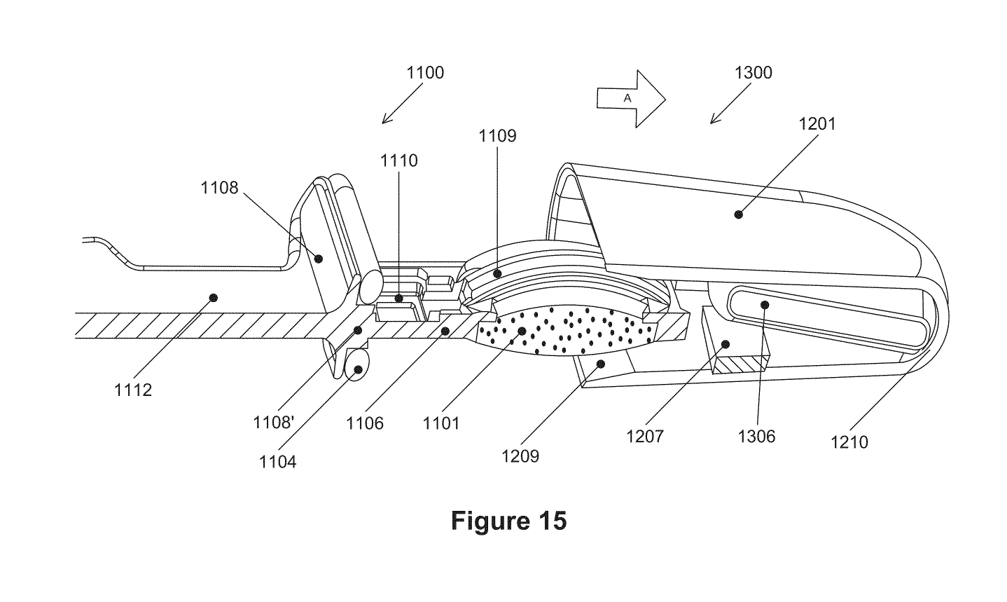

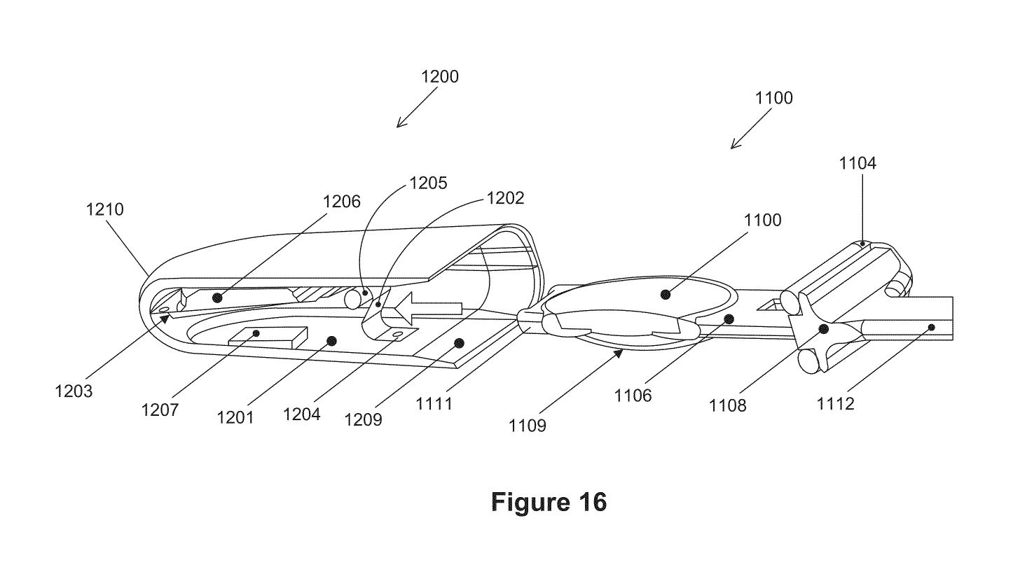

The baffle 1108 may be integrally connected to the handle 1103 and may include a curved slope abutting the neck 1112 to accommodate the general contour of a hand gripping the adjacent handle 1103 and neck 1112. The baffle 1108 may have a generally planar surface forming a side distal to the neck 1112. The baffle 1108, if positioned in intimate contact along the perimeter of an opening of a cavity (such as a user's mouth), may form a seal, separating the inside of the cavity from the outside of the cavity. Also, the baffle 1108, if positioned in intimate contact along the perimeter of an opening of the cap 1200, may form a seal, separating the inside of the cap 1200 from the outside of the cap 1200. The flat surface of the baffle 1108 may also include a ridge 1108′ extending in a distal direction as shown in FIGS. 15 and 16. The ridge may have a smaller cross section than the baffle 1108 to accommodate a compressible seal 1104. The compressible seal 1104 may be comprised of an o-ring or a gasket to assist in sealing an opening of a cavity or a cap 1200. The combination of the compressible seal 1104 and the ridge may have a combined cross-sectional width (perpendicular to the longitudinal axis B) that is substantially the same as the cross-sectional width of the outer-most portion of the baffle 1108. The compressible seal 1104 may be sized such that insertion of the distal portion of collector 1100 into a cap 1200 causes compression of the compressible seal 1104 to simultaneously establish intimate contact with the baffle 1108 and the cap 1200, forming a water-tight seal.

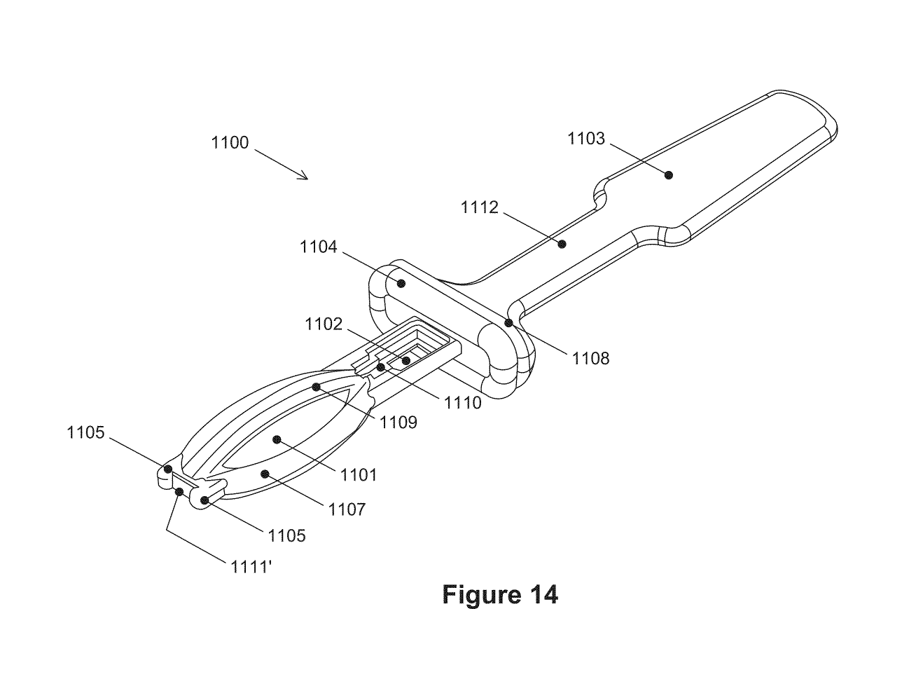

A frame 1106 having a generally rectangular cross section may extend out of the ridge 1108′ in the baffle 1108 in the distal direction. The frame 1106 may provide spacing between the absorbent pad 1101 and the baffle 1108 along the longitudinal axis B. The frame 1106 also may be integrally connected to the baffle 1108. The frame 1106 may include a sample sufficiency indicator 1110 as shown in FIGS. 14 and 15, positioned between the baffle 1108 and the absorbent pad 1101. The sample sufficiency indicator 1110 may include a reservoir to hold a volume of fluid and may also include a channel in fluid communication with the absorbent pad 1101 to transfer a volume of fluid from the absorbent pad 1101 to the reservoir. The reservoir of the sample sufficiency indicator 1110 may be connected in parallel or series with the channel. The channel may include a porous matrix (not shown) to assist in the transfer of fluid by capillary action. The sample sufficiency indicator 1110 may include a transparent window 1102 along a surface of the reservoir to provide an optical indication of a satisfactory level of fluid in the reservoir. A satisfactory level of fluid in the reservoir may be indicated by a change in the optical transmission of light. For example, if liquid displaces a volume of air in contact with the window 1102, a noticeable change in the refractive index may occur. Discerning a change in the refractive index may be aided by a pattern such as a line visible through the window 1102.

Distal to the baffle 1108, the frame 1106 may branch off into a pad holding structure 1107 to hold the absorbent pad 1101. The pad holding structure 1107 may hold an absorbent pad 1101 along a perimeter of the absorbent pad 1101 and may include a ring-like structure situated on a plane parallel to the frame 1106, having an outer perimeter and an open interior. The shape of the pad holding structure 1107 may have an elliptical or oval geometry. A rigid spoke member 1109 may connect to the pad holding structure 1107 at two opposing ends along a curved path situated on a plane orthogonal to the plane defined by the pad holding structure 1107. The rigid spoke member 1109 may assist in holding the absorbent pad 1101.

The absorbent pad 1101 may be comprised of a porous matrix having a volume configured to absorb a liquid through capillary action. The absorbent pad 1101 may have a mating shape with respect to the pad holding structure 1107. In addition, the absorbent pad 1101 may have a general convex profile. Portions of the absorbent pad 1101 may be held in place by the pad holding structure 1107 assisted by an adhesive agent. The absorbent pad 1101 may optionally be attached to the rigid spoke member 1109 by an adhesive agent. The absorbent pad 1101 may contain embedded sialagogues such as a citrus flavor or other pleasant flavors to stimulate the production of oral fluid, or may include pleasant flavors not intended to increase production of oral fluid.

An engaging section 1111 may extend out of the pad holding structure 1107 from a distal side and planar with the frame 1106. A medial portion of the engaging section 1111 may have a substantially similar cross section to the frame 1106 and may include two guides 1105 projecting outward in a direction parallel to the longitudinal axis B. The guides 1105 each may contain two opposing surfaces coplanar with two opposite sides of the frame 1106. Each of the guides 1105 may have a cross section defined in part by a semicircle with one endpoint of each semicircle being coincident with an outer surface of the engaging section 1111; the other endpoints coincident with a recessed surface 1111′. The recessed surface 1111′ may be a planar surface orthogonal to the longitudinal axis B.

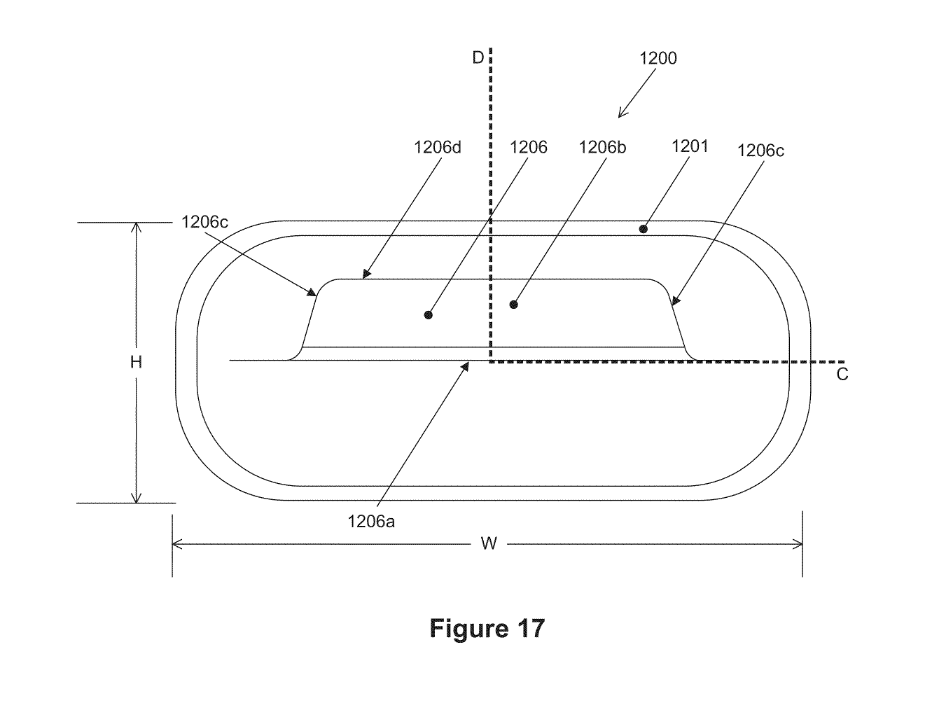

As shown in FIGS. 13, 16 and 17, the cap 1200 may contain a shell 1201 sharing the longitudinal axis B of the collector 1100. A cross section orthogonal to longitudinal axis B may reveal a major axis C and a minor axis D, the major axis C corresponding to the direction of the width W of the shell 1201 along the cross section, and the minor axis D corresponding to the direction of the height H within the same plane, and orthogonal to the major axis C. The shell 1201 may form an enclosure having a planar opening at one side orthogonal to the longitudinal axis B. The shell 1201 may include a sloping entry area 1209 having a substantially constant thickness along its perimeter. The thickness of the shell 1201 in the sloping area 1209 may increase in the direction opposite to the opening. The remaining portion of the shell 1201 may have a substantially constant thickness. The width W and the height H of the shell 1201 may decrease along the longitudinal axis B to an end portion 1210. The shell 1201 may include two substantially planar sides parallel to the major axis C, each of the sides forming a symmetrical acute angle with respect to the longitudinal axis B. The shell 1201 may include two substantially planar sides parallel to the minor axis D, each of the sides forming a symmetrical acute angle with respect to the longitudinal axis B. A cross section of the shell 1201 orthogonal to the longitudinal axis B may reveal a plurality of rounded corners, each sharing a contiguous edge between a planar side parallel to the major axis C and a planar side parallel to the minor axis D. The rounded corners may form a surface of the shell 1201 beginning at the opening of the shell 1201 and sweeping along a curvilinear path, and returning to the opening of the shell at an opposite side, forming a reflective symmetry across the minor axis D. An end tip 1210 of the shell 1201 may be located opposite to the opening of the shell 1201. The shell 1201 may also include a keying member (not shown) configured to allow the collector 1100 to be inserted in a preferred orientation. The shell 1201 may also include guide members (not shown) to assist in aligning the end of the collector 1100 if inserted into the cap 1200.

The cap 1200 may include a reservoir 1206 for holding fluid comprising reagents. The reservoir 1206 may have a bottom planar surface 1206a having a perimeter. The perimeter may include a first section configured to be substantially contiguous with an inner surface of the cap 1200 and a second section configured to be substantially parallel with the opening of the shell 1201 when the reservoir 1206 is positioned towards the end tip 1210. The reservoir 1206 may include an outward protrusion 1206b from the bottom planar surface 1206a having a base perimeter defined by a constant inward offset from the perimeter of the bottom planar surface 1206a. The outward protrusion 1206b may include sloping vertical walls 1206c with respect to the bottom planar surface 1206a defining a top planar surface 1206d having a top perimeter defined by an inward offset from the base perimeter. The sloping walls 1206c may further include a fillet at their interface with the bottom planar surface 1206a and a rounded corner at their interface with the top planar surface 1206d. The bottom planar surface 1206a may be further positioned within the cap 1200 such that the bottom planar surface 1206a is substantially coincident with a plane defined by the longitudinal axis B and the major axis C. The reservoir 1206 may be comprised of materials that are the same as or similar to the materials comprising reservoir 130. The reservoir 1206 may define a cavity for holding a fluid comprising a reagent. The bottom planar surface 1206a of the reservoir 1206 may also include a flexible film 1203 comprising a substantial portion of the bottom planar surface 1206a and a tail portion 1204 extending in a general direction towards the opening of the shell 1201. A cross section of the tail portion 1204 may have a width parallel to the major axis C and may be configured to be smaller than the spacing of the guides 1105 of the engaging section 1111. The tail portion 1204 may be attached to the shell 1201 along an inner surface opposing the bottom planar surface 1206a of the reservoir 1206 at an attachment site. The attachment site may be located at the intersection of the shell 1201 and a plane defined by the longitudinal axis B and the minor axis D. The film 1203 may be configured to separate from the bottom planar surface 1206a of the reservoir 1206 upon receiving a sufficient force. A sufficient force may be defined as a force that a user is capable of supplying with one of his/her hands. The reservoir 1206 may be configured to be water-tight until disruption or separation of the film 1203.

The cap 1200 may include a crossbar 1205 (FIG. 16) having a cylindrical cross section with its axis parallel to the major axis C. The crossbar 1205 may connect to two sidewalls of the shell 1201 at a location further towards the opening of the shell 1201 with respect to the reservoir 1206. The crossbar 1205 may also be positioned in an upper half of the shell 1201, in the half containing the reservoir 1206. The medial plane of the upper and lower halves may be defined by the longitudinal axis B and the major axis C. The tail portion 1204 may be routed from the edge of the bottom planar surface 1206a of the reservoir 1206 to the attachment site on the shell 1201 along a path above the crossbar 1205.

The cap 1200 may include a test results indicator 1207 located on the lower half of shell 1201. The test results indicator 1207 may include a window through the shell 1201 exposing an assay device under the window, and may also expose any results associated with the assay device. The assay device may include lateral flow and flow-through detection devices and may determine the presence and/or amount of an analyte in a liquid sample as the liquid sample moves through the assay device by lateral flow or capillary action. The assay device may be used in a vertical or a horizontal orientation or in an orientation between vertical and horizontal. The assay device may contain materials configured to change color in the presence of an analyte.

The user may remove the collector 1100 and cap 1200 from a single packaging container (not shown). The user may then grasp the handle 1103 and neck 1112 with one of their hands and direct the absorbent pad 1101 inside their mouth. The user may swab (or hold in place) the absorbent pad 1101 along the mucosal tissue in their mouth including their gums, tongue, beneath their tongue, and the inside of their cheeks or in the interior of their mouth. The user may perform the swabbing with their mouth open or closed and allow oral fluid to collect on the absorbent pad 1101.

Oral fluid may flow from the absorbent pad 1101 to a sample sufficiency indicator 1110 through a porous matrix (not shown) contained within a channel of the frame 1106. Oral fluid may then collect within a reservoir of the sample sufficiency indicator 1110 until a visual indication of a sufficient amount of oral fluid is present. As oral fluid fills the reservoir in the sample sufficiency indicator 1110, a noticeable change in the refractive index of light may occur. Discerning a change in the refractive index of light may be aided by a pattern such as a line visible through the window 1102. After a sufficient amount of oral fluid is indicated, the user may then remove the end of the collector 1100 comprising the absorbent pad 1101 from their mouth.

Next, the user may insert the distal end of the collector 1100 into the cap 1200 along the longitudinal axis B in a direction A as shown in FIG. 16. The distal end of the collector 1100 may be aligned by the guide members (not shown) of the shell 1201 to direct the engaging section 1111 to engage the tail portion 1204 in the recess formed by the guides 1105. Contact between the engaging section 1111 and the tail portion 1204 permits the absorbent pad 1101 to be inserted fully into the cap 1200 without being substantially compressed and also while substantially retaining its volume. Further advancement of the absorbent end may create tension in the tail portion 1204 and foil 1203. When the tension exceeds a predetermined value, the foil 1203 may separate from the bottom planar surface 1206a of the reservoir 1206 and provide an opening for reagents to flow out of the reservoir 1206. As the absorbent end is advanced further in the direction A, the foil 1203 may move in a retrograde motion, further separating the foil 1203 from the bottom planar surface 1206a of the reservoir 1206. As the engaging member 1111 approaches the end tip 1210, the compressible seal 1104 may start to make contact with the sloping area 1209. Additional force applied in direction A may cause the absorbent end to lock into place with the absorbent pad 1101 positioned adjacent to the reservoir 1206, and establish a water-tight seal between the compressible seal 1104 and the sloping area 1209. In the locked position, the cap 1200 and the interior walls of the shell 1201, baffle 1108, and the seal 1104 may define a chamber with a fixed volume. Locking of the collector 1100 to the cap 1200 may be accompanied by an audible and/or tactile indication notifying the user that both the collector 1100 may be locked to the cap 1200 and that a water-tight seal may exist between the collector 1100 and the cap 1200.

After the collector 1100 and the cap 1200 are sealed, reagents from the reservoir 1206 may come into contact with the oral fluid in the absorbent pad 1101 through a diffusion or lateral flow process. The reagents may facilitate the transport of both of enzymes, antibodies and/or substrates within the oral fluid out of the absorbent pad 1101 to a test results indicator 1207, similar to test results indicator 143, without a substantial change in the volume of the absorbent pad 1101.

The test results indicator 1207 may include within separately spaced designated test zones, similar to test zones 144: an immobilized HIV-specific antigen such as P24, a control antigen, and other control compounds such as a paired HIV-specific antibody/antigen. If an HIV-specific antibody is present in the oral fluid, it may bind to the HIV-specific antigen and the associated enzyme may react with the conjugate pair to produce a color indication of the presence of the HIV-specific antibody. At a different location within the test results indicator 1207, the enzyme may react with a paired HIV-specific antibody/antigen to produce a color indicating a positive control. At an even different location within the test results indicator 1207, a control antibody may bind to a control antigen and an associated enzyme may react with the conjugate pair to produce a color indicating a negative control. Based upon the indications from the negative and/or positive controls, the user may then acknowledge that the test results are ready. The user may then optionally repackage the collector 1100 and cap 1200 in a separately provided package (not shown) to transport or send to a testing facility to perform additional testing on the collected and sealed sample.

In another embodiment, the cap 1200 is replaced with cap 1300 as shown in FIG. 15. Cap 1300 may include the elements of cap 1200 as disclosed above, with the exception that the reservoir 1206 may be replaced with reservoir 1306, and the following elements may be omitted: crossbar 1205, foil 1203, and tail portion 1204.

Cap 1300 may include a reservoir 1306 for holding a fluid containing a reagent. The reservoir may be comprised of a glass ampoule (or ampoules) having a substantially cylindrical profile. The longitudinal axis of the reservoir 1306 may form an inclination angle relative to the longitudinal axis B. The reservoir 1306 may be fixed in place with respect to the shell 1201. The reservoir 1306 may be comprised of materials similar to reservoir 130.

In another embodiment, the method of using the collector 1100 with the cap 1300 may include the same steps as using the collector with the cap 1200 as described above; however, the process of discharging the fluid from inside the reservoir 1306 will be provided in more detail.

The user may insert the distal end of the collector 1100 into the cap 1300 along the longitudinal axis B in a direction A as shown in FIG. 15. The distal end may be aligned by the guide members of the shell 1201 (not shown) to direct the engaging section 1111 to engage the reservoir 1306 in the recess formed by the guides 1105. Advancement of the absorbent end may direct the rigid spoke member 1109 underneath the reservoir 1306. Further advancement of the absorbent end may create tension in the reservoir 1306, by the rigid spoke member 1109 and/or the recess formed by the guides until the tension exceeds a predetermined value, at which point the reservoir may crack, shatter, and/or burst, and create an opening for reagents to flow out. Contact between the rigid spoke member 1109 and the reservoir 1306 permits the absorbent pad 1101 to be inserted fully into the cap 1200 without being substantially compressed and also while substantially retaining its volume. As the engaging member 1111 approaches the end tip 1210, the compressible seal 1104 may start to make contact with the sloping area 1209. Additional force applied in direction A may cause the absorbent end to lock into place and establish a water-tight seal between the compressible seal 1104 and the sloping area 1209. Locking of the collector 1100 to the cap 1300 may be accompanied by an audible and/or tactile indication notifying the user that both the collector 1100 may be locked to the cap 1300 and that a water-tight seal may exist between the collector 1100 and the cap 1300.

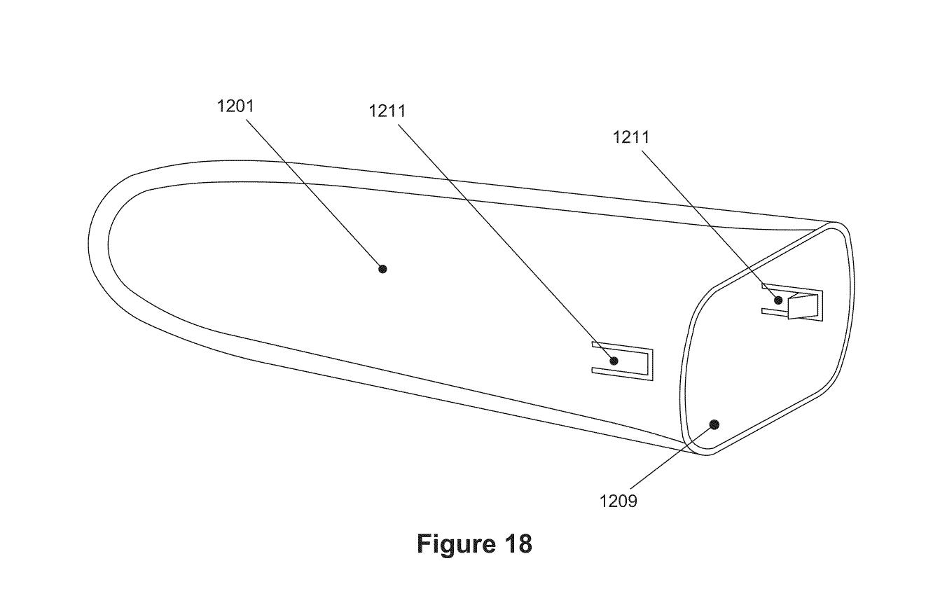

In another embodiment shown in FIG. 18, the caps 1200 and 1300 may include elastic snaps 1211 along the inner surface of the shell 1201 having a slanting surface forming an angle with the longitudinal axis B and having a flat surface orthogonal to the longitudinal axis B. As the distal end of the collector 1100 is inserted into the caps 1200 and 1300 in the direction A, the seal may contact the snaps 1211. As the distal end is further advanced in the direction A, the snaps 1211 may flex such that the angle formed by the slanted surface increases with respect to the longitudinal axis B. Once the compressible seal 1104 and the baffle 1108 are advanced beyond the snaps 1211, the snap 1211 may elastically return to its previous position, such that the flat surface makes contact with the handle-side of the baffle 1108, locking the collector 1100 in place with respect to the caps 1200 and 1300. The shell 1201 may include slots to allow the snaps 1211 to flex in a direction away from the collector 1100 when inserted. The snaps 1211 may be configured to release the collector 1100 by simultaneously flexing the snaps 1211 in a direction away from the longitudinal axis B, while the collector 1100 is retreated in an opposite direction of A. The snaps 1211 may provide an audible and/or tactile indication when the collector 1100 is locked in place.

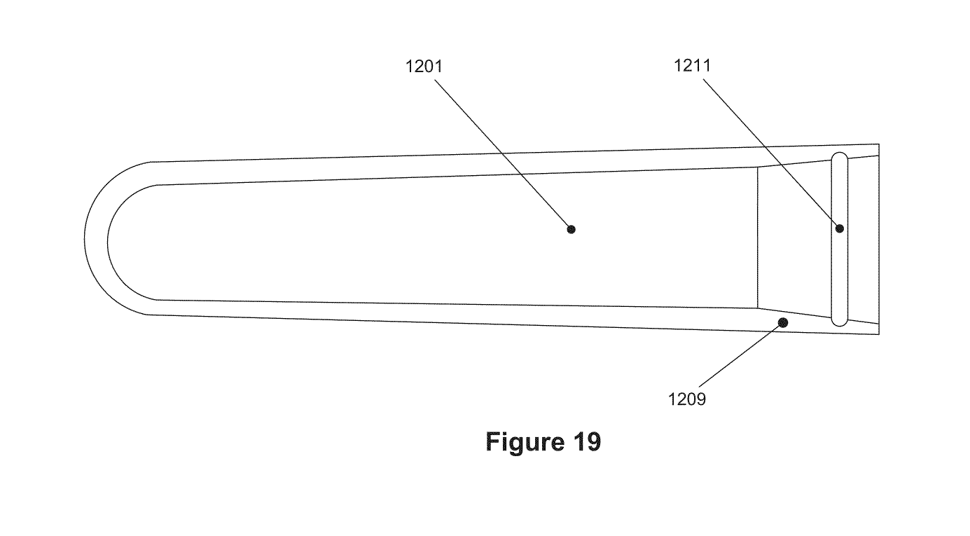

In another embodiment shown in FIG. 19, the sloping area 1209 may include a circumferential recess 1212 having a mating profile with respect to an outer perimeter of the baffle 1108 and configured to lock the collector 1100 in place with respect to the caps 1200 and 1300. The recess may provide an audible and/or tactile indication when the collector 1100 is locked in place.

In other embodiments, the collector 1100 and the caps 1200 and 1300 may be constructed with various materials and techniques. For example, the handle 1103, baffle 1108, frame 1106, pad holding structure 1107, rigid spoke member 1109, engaging section 1111, guides 1105, shell 1201, sloping area 1209, and crossbar 1205 may be comprised of similar materials as housing 110 and shield 120.

Additionally, the absorbent pad 1101 may be attached to the pad holding structure 1107 by other fastening modalities such as tongue and groove, clamping, or hook and loop.

In other embodiments, the shell 1201 may include a transparent material, and may obviate the need for a window to view the test results indicator 1207. In this embodiment, the test results may be visible through the transparent shell 1201.

In other embodiments, the window to the test results indicator 1207 and/or the window 1102 may include a water-tight transparent film.

In other embodiments, a reservoir including a reagent may be integrally molded with the shell 1201.

In other embodiments, the sample sufficiency indicator 1110 may include an expansion member capable of expanding upon contact of a fluid. The expansion member may include a sponge or a super absorbent polymer such as sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. A user may determine that a sufficient amount of fluid has entered the reservoir of the sample sufficiency indicator 1110 based upon optical recognition of the expansion of the expansion member. Alternatively, the sample sufficiency indicator 1110 may include a porous matrix having color changing salts embedded, such as cobalt chloride. As the cobalt chloride becomes saturated, an optical change in color may be observed. Alternatively, the sample sufficiency indicator 1110 may include circuit elements capable of measuring a property that may be affected by a saturated film, including measuring the capacitance or resistance of such a film. A change in resistance or capacitance due to the presence of a sample may be indicated optically with a light source such as an LED or audibly with a sound source such as a speaker or beeper.

In other embodiments, the test results indicator 1207 may include circuit elements capable of measuring a quantifiable degree of a positive test result. This quantifiable degree may be compared to predetermined levels to label the result into categories. The test results indicator may further include a display such as an LED array or LCD to reveal the label of the result. The label may be a characterizing word or phrase representative of the category and may include “positive,” “negative,” “retest,” “borderline,” “uncertain,” “indeterminate,” and/or “see your doctor.” The display may include backlighting to assist the user in reading the results in a poorly lit environment.

In other embodiments the reservoir 1206 may be opened with an electric signal such as an actuator. The reservoir 1206 may also be opened by a change in temperature such as electrically heating a wire embedded within a heat sensitive membrane, such as a gelatin membrane.

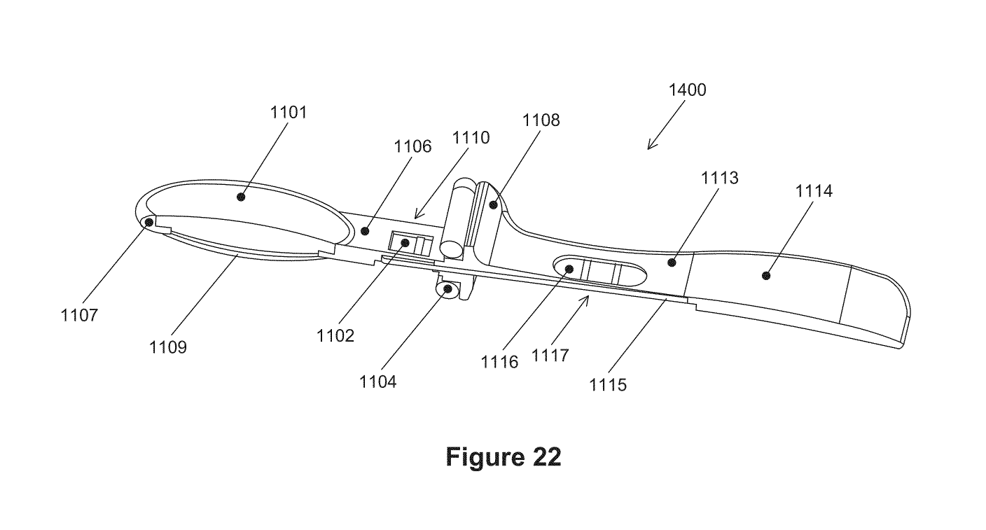

In another embodiment shown in FIG. 22, a collector 1400 with a test results indicator 1117 is disclosed. The collector 1400 may include many of the same features as the collector 1100, particularly the absorbent pad 1101, transparent window 1102, compressible seal 1104, frame 1106, pad holding structure 1107, baffle 1108, rigid spoke member 1109, and the sample sufficiency indicator 1110. The collector 1400 may include a neck 1113 and handle 1114 similar to the neck 1112 and handle 1103 in form and function with cosmetic changes. The neck 1113 may also include a test results indicator 1117 further including a window 1116. The window 1116 may allow optic inspection of a lateral test flow device 1115 configured to indicate the presence of an antibody during a test. The antibody may further include an antibody associated with HIV. The collector 1400 in conjunction with cap 1200 or 1300 may function in a similar manner to collector 1100 in conjunction with cap 1200 or 1300 and may include the following additional features. The collector 1400 may include a channel in fluidic communication from the absorbent pad 1101 through the frame 1106, baffle 1108 and neck 1113 to the handle 1114. The channel may include a porous matrix 1115 visible through windows 1102 and 1116. The porous matrix 1115 may facilitate the transfer of oral fluid to the oral fluid sufficiency indicator 1110. Through the oral fluid sufficiency indicator 1110, the user may be able to make a determination if a sufficient amount of oral fluid is present in a similar manner as with the collector 1100. In addition, the porous matrix 1115 may facilitate the transfer of reagents and antibodies to the test results indicator 1117. The test results indicator 1117 may assist the user in making a determination if HIV-specific antibodies are present in the oral fluid in a similar manner as with the test results indicator 1207 as discussed previously.

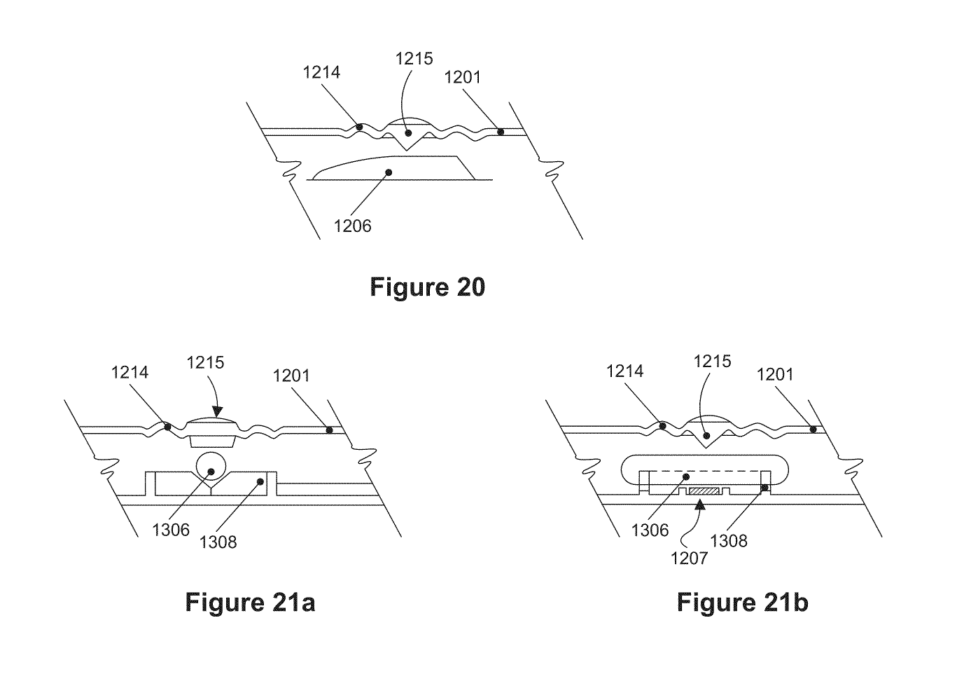

Other embodiments may further reduce the risk of spilling the reagents by including an additional step of opening the reservoirs 1206 and 1306 once the collector 1100 and cap have established a seal. In an embodiment shown in FIG. 20, the reservoir 1206 may be punctured externally with respect to the collector 1100 and a cap to release fluid that it may enclose. In this embodiment, the shell 1201 may include a push button 1215 located along the longitudinal axis B. The push button 1215 may include a generally chisel-shaped structure positioned over the proximal end of the reservoir 1206. The upper surface of the push button 1215, may include a convex profile. The upper surface of the shell 1201 may include a flexure 1214 including a corrugated pattern surrounding the push button 1215. The flexure 1214 may include a series of crests and troughs each concentric with a circular top of the push button 1215. The flexure 1214 may be configured to flex in such a way to allow the push button to move in parallel with the minor axis D. After the collector 1100 is inserted into a cap, the push button 1215 may be pressed in a direction towards the inside of the cap in parallel with the minor axis D. The bottom surface of the push button may be pressed until the reservoir 1206 is punctured, causing fluid inside the reservoir 1206 to be expelled from reservoir 1206 and into the cavity of the shell 1201.

In an embodiment shown in FIGS. 21a and 21b, the reservoir 1306 may be punctured externally with respect to the collector 1100 and a cap, similar to caps 1200 and 1300, to release fluid that it may enclose. Similarly with the embodiment shown in FIG. 8, the shell 1201 may include a push button 1215 located along the longitudinal axis B. The push button 1215 may include a generally chisel-shaped structure positioned over the proximal end of the reservoir 1306. The upper surface of the push button 1215, may include a convex profile. The upper surface of the shell 1201 may include a flexure 1214 including a corrugated pattern surrounding the push button 1215. The flexure 1214 may include a series of crests and troughs each concentric with a circular top of the push button 1215. The flexure 1214 may be configured to flex in such a way to allow the push button to move along minor axis D. After the collector 1100 is inserted into a cap, the push button 1215 may pressed in a direction towards the inside of the cap along the minor axis D. The bottom surface of the push button may be pressed until the reservoir 1306 is cracked or shattered, causing fluid inside the reservoir 1306 to be expelled from reservoir 1306 and into the cavity of the shell 1201.

In another embodiment, the crossbar 1205 and/or guides within the shell 1201 may be configured to squeegee fluid out of the absorbent pad 1101 upon advancement of the absorbent end into the caps 1200 and 3100. As the distal end of the collector 1100 is inserted into the caps 1200 and 1300 along the longitudinal axis B, a surface of the pad supporting structure 1107 may make contact or come into close proximity to the cross bar 1205 such that the absorbent pad 1101 may compress and expel fluid.

In another embodiment, the reservoirs 1206 and 1306 may be opened after the caps 1200 and 1300 are sealed. The engaging member 1111 and/or the rigid spoke member 1109 may make contact with the reservoir 1306 or the tail portion 1204 after the compressible seal 1104 makes contact with the sloping area 1209. Fluid may leave the reservoirs 1206 and 1306 after the seal has been established between the collector 1100 and the caps 1200 and 1300. The fluid may become contained in the caps 1200 and 1300 preventing leaking and allowing the insertion of the collector 1100 into the caps 1200 and 1300 to occur in any orientation without the potential for leaking during the insertion.

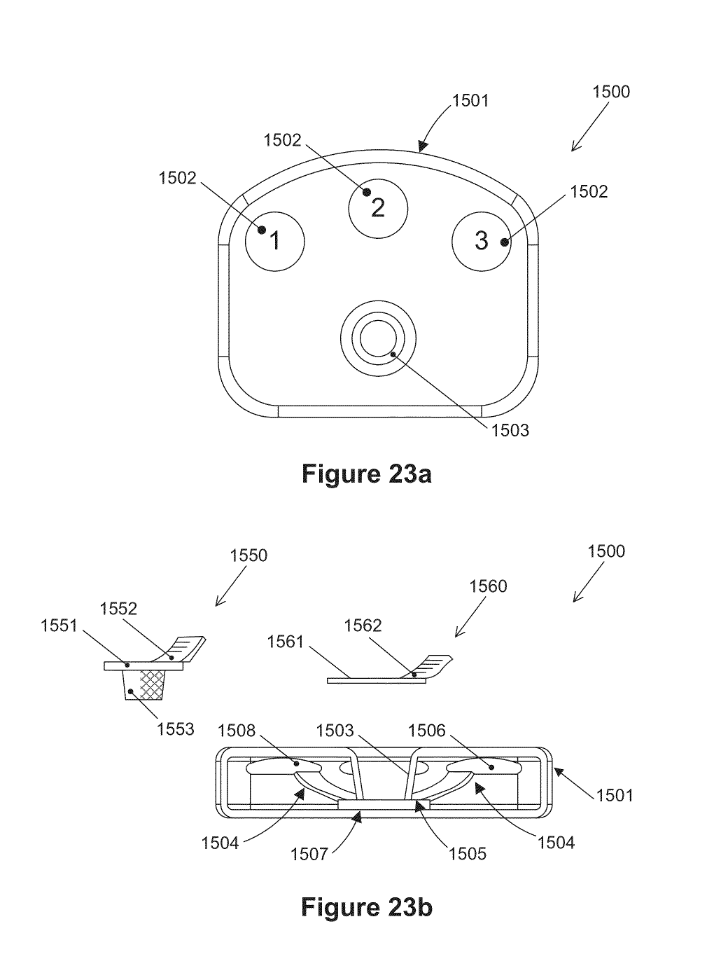

Other embodiments may allow reagents to be released from multiple sources at various times during a test. In an embodiment shown in FIGS. 23a and 23b, a collector 1500 may include a shell 1501 enclosing a cavity and defining a funnel-shaped opening 1503. The collector 1500 may include one or more reservoirs 1506, each of which may further include a reagent. Each reservoir 1506 may be in fluid communication with a results indicator 1507 by a channel 1504. The collector 1500 may include a membrane 1505 positioned between the results indicator 1507 and the opening 1503. The opening 1503 may include a removable filter 1550 and a cover 1560. The filter 1550 may include a filter element 1553 connected to a support disk 1551. The support disk 1551 may be connected to a filter handle 1552 to allow a user to grip and move the filter 1550. The cover 1560 may include a flat surface 1561 configured to cover the disk 1551 and the opening 1503. The flat surface 1561 may include an adhesive on at least its perimeter to seal the cover 1560 to the shell 1501. The cover 1560 also may include a handle 1562 to allow a user to grip and move the cover 1560. The collector 1500 may include one or more buttons 1502 on the outside of the shell 1501. Each button 1502 may be configured to open one of the reservoirs 1506 to allow a reagent to flow to the results indicator 1507. The buttons 1502 may be labeled with numbers or other indicia to indicate a predetermined sequence and additionally the buttons 1502 may be coded by different colors. For example the three different buttons 1502 may be labeled 1, 2, 3, and/or colored green, yellow, and red respectively mimicking the color sequence at a traffic light. The results indicator may function in a similar manner as the previously described results indicator 1107.

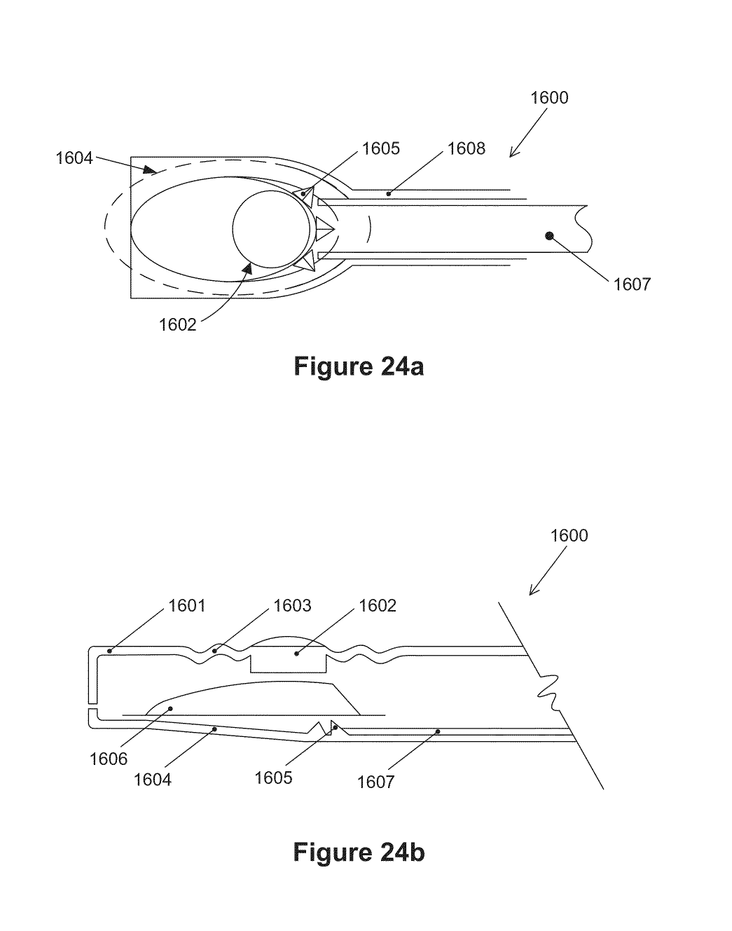

More detail will be drawn to other embodiments of a button release mechanism. FIGS. 24a and 24b show a pouch-releaser 1600 according to an exemplary embodiment. The pouch-releaser 1600 may include a shell 1601 forming a cavity. The shell 1601 may include an integrally molded flexure 1603. The flexure 1603 may include a series of wave-like corrugations concentrically surrounding a button 1602. The button 1602 may have a substantially cylindrical profile with a convex upper surface. The flexure 1603 may be configured to flex in a direction parallel with the axis of the button 1602, allowing the button 1602 to move inward within the cavity of the shell 1601. The pouch-releaser 1600 may include a reservoir 1606 positioned above a sloping portion 1604 of the shell 1601. The sloping portion 1604 may be in fluidic communication with a channel 1608. The channel 1608 may be configured to transport a fluid and may include a porous matrix 1607 to assist in transporting the fluid. The sloping well 1604 and/or the channel 1608 may contain teeth 1605 arranged in an arc concentric with the button 1602, having a slightly larger diameter. When a user presses down on the button 1602, a surface of the reservoir 1606 may be forced into the teeth 1605 rupturing the reservoir 1606 and allowing reagent to flow onto the sloping well 1604 and through the channel 1608.

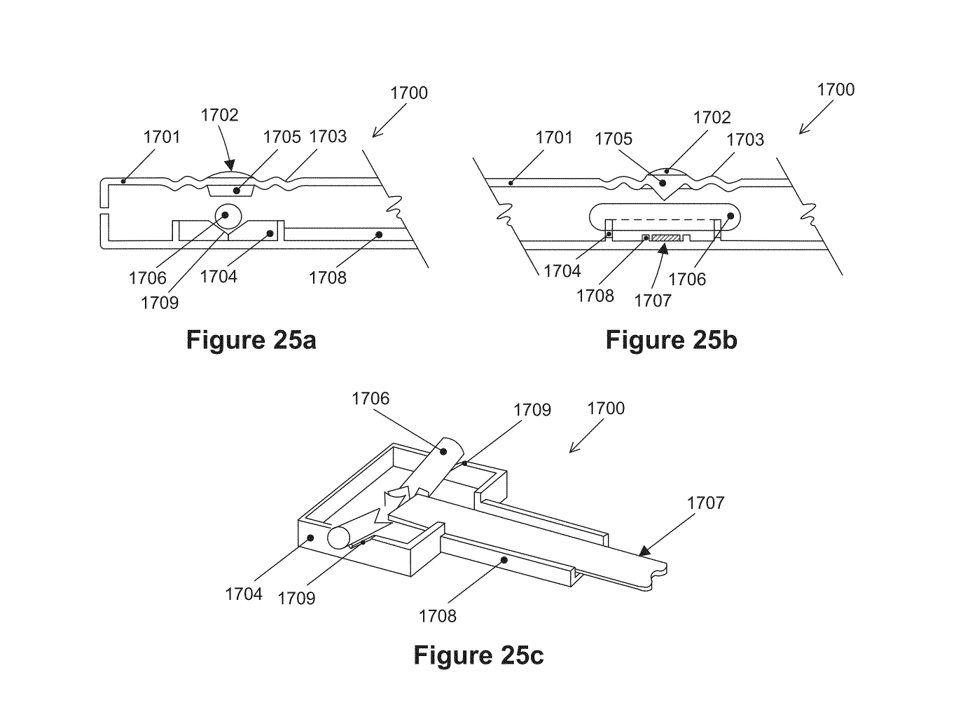

FIGS. 25a, 25b, and 25c show an ampoule-releaser 1700 according to an exemplary embodiment. The ampoule-releaser 1700 may include a shell 1701 forming a cavity. The shell 1701 may include an integrally molded flexure 1703. The flexure 1703 may include a series of wave-like corrugations concentrically surrounding a button 1702. The button 1702 may have a circular convex upper surface and a chisel-like bottom surface 1705 positioned above a middle portion of a reservoir 1706, similar to reservoir 1206. The flexure 1703 may be configured to flex in a direction orthogonal to the top surface of the shell 1701, allowing the button 1702 to move inward within the cavity of the shell 1701. The reservoir 1706 may be positioned in a well structure 1704. The well structure 1704 may include an upwardly directed wall defining at least two recesses 709 configured to hold the reservoir 1706 beneath the button 1702. The well structure 1704 may be in fluidic communication with a channel 1708. The channel 1708 may be configured to transport a fluid and may include a porous matrix 1707 to assist in transporting the fluid. When a user presses down on the button 1702, a chisel-like bottom surface 1705 may be forced into the reservoir 1706 and may crack or shatter the reservoir 1706 allowing reagent to flow onto the well structure 1704 and through the channel 1708.

Advantages of the aforementioned embodiments may become readily apparent. At least some of the embodiments may allow a user to perform an HIV test (or a different test) in privacy with the test results available very soon after a sample of bodily fluid is collected, e.g., within seconds or minutes. This may be accomplished by not having to transfer a sample to a test facility, wait for the test facility to analyze the sample, and wait for the test results to be transferred or communicated back. This also may obviate the need to have the test performed by a health care professional in a traditional health care setting such as a doctor's office, hospital or health clinic. The test may be performed under low lighting conditions. One of ordinary skill would be able to contemplate the many locations and circumstances in which a test may be performed. For example, the test may be a home test, wherein the term “home test” may include any location of a user's choosing, at the place and time of one's choosing, including in the home, in a hotel/motel room, in a bar, in a bath house, or at a point-of-care. The user may be able to perform a test before engaging in an activity that presents conditions for an increased risk of transferring/contracting, e.g., HIV, such as at the point-of-sex (immediately before anticipating engaging in sexual activity).

Further advantages of at least some of the embodiments may include small size, ease of use, reduced risk of user test errors, and therefore less chance of an unreliable test result. In some examples, the small size of the device may allow it to fit inconspicuously in a purse or pocket, making it easy to transport. Including prepackaged reagents may minimize the chance of spilling (which is a recognized source of error in certain types of other tests) and exposure to contamination, thus reducing the user's anxiety. In some embodiments, the user may release the reagents with the push of a button. This may reduce time that may otherwise be spent opening a reagent container, measuring reagents, and skillfully transferring the reagents. Also, in some embodiments, the user may observe the results of the test while the assay device is held at any orientation.

Additional advantages of some embodiments of the device include allowing for testing for HIV specific antigens. HIV specific antigens, HIV viral RNA, DNA, and reverse transcriptase or other markers of HIV infection may be present in bodily fluids at higher concentrations than the HIV specific antibodies soon after contracting the virus, and may exist before detectable levels of HIV antibodies are even present. This may allow for the detection and treatment at an earlier time period compared to the detection of HIV antibodies only. An individual is considerably more infectious in the time period shortly after contracting HIV, thus society as a whole would benefit from early detection. Also, one of ordinary skill would appreciate the increase in sensitivity and reliability gained by testing for HIV antigens in addition to testing for just HIV antibodies.

Other embodiments of the invention will be apparent to those skilled in the art from consideration of the specification and practice of the invention disclosed herein. It is intended that the specification and examples be considered as exemplary only.

What is claimed is:

1. A test device for detecting an analyte in a sample of bodily fluid, comprising:
at least one hollow needle configured to obtain a sample of bodily fluid;
an enclosure configured to contain a reagent;
a portion configured to open the enclosure;
a test results indicator configured to absorb the bodily fluid, the test results indicator including at least one test zone configured to indicate the presence of an analyte in the bodily fluid when the at least one test zone is in fluidic contact with the reagent;
a housing; and
a cover configured to be movable with respect to the housing between a closed position precluding access to the at least one hollow needle and an open position permitting access to the at least one hollow needle;
wherein the at least one hollow needle is in fluid communication with the test results indicator.

2. The test device according to claim 1, wherein the test results indicator is configured to indicate the presence of HIV.

3. The test device according to claim 1, wherein the at least one hollow needle has a coating comprising an anesthetic.

4. The test device according to claim 1, wherein the at least one hollow needle has a longitudinal length equal to or less than 4 mm.

5. The test device according to claim 1, wherein the test results indicator is a first test results indicator configured to indicate the presence of a first analyte in the bodily fluid, and wherein the test device further comprises a second test results indicator configured to absorb the bodily fluid, the second test results indicator including at least one second test zone configured to indicate the presence of a second analyte in the bodily fluid when the at least one second test zone is in fluidic contact with the reagent, the second analyte differing from the first analyte; and
wherein the first test results indicator is spaced apart from the second test results indicator.

6. The test device according to claim 5, further comprising a first fluid transport portion between the at least one hollow needle and the first test results indicator, and a second transport portion between the at least one hollow needle and the second test results indicator.

7. The test device according to claim 6, wherein the first fluid transport portion is configured to transport a larger volume of the bodily fluid than the second fluid transport portion.

8. The test device according to claim 1, wherein the cover is configured to be in the open position while the enclosure is open.

9. A test device for detecting an analyte in a sample of bodily fluid, comprising:
a sample collector defining a first volume configured to contain a sample of bodily fluid;
an enclosure configured to contain a reagent;
a portion configured to open the enclosure;
a chamber defining a fixed second volume, the chamber being configured to permit mixing of the bodily fluid and the reagent;
a test results indicator configured to absorb the bodily fluid, the test results indicator including at least one test zone configured to indicate the presence of an analyte in the bodily fluid when the at least one test zone is in fluidic contact with the reagent; and
wherein the sample collector is in fluid communication with the test results indicator such that the bodily fluid flows from the sample collector to the test results indicator; and
wherein the sample collector is configured such that the first volume remains substantially unchanged when bodily fluid flows from the sample collector to the test results indicator.

10. The test device according to claim 9, wherein the test results indicator is configured to indicate the presence of HIV.

11. The test device according to claim 9, wherein the sample collector includes at least one hollow needle.

12. The test device according to claim 11, wherein the at least one hollow needle has a longitudinal length equal to or less than 4 mm.

13. The test device according to claim 11, wherein the sample collector includes an opening defining a cavity having an interior bottom portion, the interior bottom portion including a raised section;
wherein the at least one hollow needle extends out from the bottom portion by a first height;
wherein the raised section has a second height greater than or equal to the first height; and
wherein at least a portion of the raised section is positioned between the at least one hollow needle and the opening.

14. A method for detecting an analyte in a sample of bodily fluid via a test device including
a sample collector defining a volume configured to contain a sample of bodily fluid,
an enclosure containing a reagent, and
a test results indicator configured to absorb the bodily fluid, the test results indicator including at least one test zone configured to indicate the presence of an analyte in the bodily fluid when the at least one test zone is in fluidic contact with the reagent,
wherein the method comprises:
acquiring the sample of bodily fluid with the sample collector;
conveying the bodily fluid from the sample collector;
releasing the reagent from the enclosure; and
mixing the bodily fluid with the reagent;
wherein the volume of the sample collector remains substantially unchanged during the releasing the reagent.

15. The method according to claim 14, wherein the sample collector includes at least one hollow needle; and
wherein acquiring the sample further includes piercing tissue via the at least one hollow needle such that the sample flows through the at least one hollow needle.

16. The method according to claim 15, wherein the at least one hollow needle has a longitudinal length equal to or less than 4 mm.

17. The method according to claim 14, wherein the test device further includes a cover and a housing; and
the method further includes locking the cover to the housing after the acquiring the sample.

18. The method according to claim 14, wherein the mixing the bodily fluid with the reagent occurs while the test device is positioned in any horizontal or vertical orientation.

19. The method according to claim 14, wherein the acquiring the sample and the releasing the reagent occur at substantially the same time.

20. The method according to claim 14, wherein the method further includes indicating the presence or absence of the analyte in the bodily fluid.
